(19) United States
(12) Patent Application Publication
(10) Pub. No.: US 2025/0064983 A1
(43) Pub. Date: Feb. 27, 2025

(54) COMPOSITIONS AND METHODS FOR TREATING A CARDIAC DISEASE

(71) Applicant: The Children's Medical Center Corporation, Boston, MA (US)

(72) Inventors: Vassilios J. Bezzerides, Boston, MA (US); William J. Pu, Boston, MA (US); Sofia M. De La Serna Buzon, Boston, MA (US)

(73) Assignee: The Children's Medical Center Corporation, Boston, MA (US)

(21) Appl. No.: 18/948,738

(22) Filed: Nov. 15, 2024

Related U.S. Application Data
(63) Continuation of application No. PCT/US2023/022269, filed on May 15, 2023.
(60) Provisional application No. 63/342,311, filed on May 16, 2022.

(51) Int. Cl. A61K 48/00; A61P 9/06; C07K 14/00; C12N 15/86

(52) U.S. Cl. CPC A61K 48/0058; A61K 48/0033; A61P 9/06; C07K 14/00; C12N 15/86; C12N 2750/14143

(57) ABSTRACT

The invention of the disclosure features Ca²⁺-calmodulin dependent kinase II (CaMKII) inhibitory multimeric polypeptides polynucleotides encoding such polypeptide, and methods of using the same for the treatment of cardiac diseases (e.g., catecholaminergic polymorphic ventricular tachycardia (CPVT) or atrial fibrillation (AF)).

### CROSS-REFERENCE TO RELATED APPLICATIONS

This application is a continuation under 35 U.S.C. § 111(a) of PCT International Patent Application No. PCT/US2023/022269, filed May 15, 2023, designating the United States and published in English, which claims priority to and the benefit of U.S. Provisional Application No. 63/342,311, filed May 16, 2022, the entire contents of which are incorporated by reference herein.

### STATEMENT OF RIGHTS TO INVENTIONS MADE UNDER FEDERALLY SPONSORED RESEARCH

This invention was made with government support under Grant No. W81XWH1910473 awarded by the Department of Defense. The government has certain rights in the invention.

### SEQUENCE LISTING

The present application contains a Sequence Listing which has been submitted electronically in XML format. The entire contents of the electronic XML Sequence Listing (Date of creation: Oct. 3, 2023; Size: 17,185 bytes; Name: 167705-031101_PCT_SL.xml).

### BACKGROUND OF THE INVENTION

Catecholaminergic polymorphic ventricular tachycardia (CPVT) is a condition characterized by an abnormal heart rhythm, which affects as many as one in ten thousand people. Symptoms of CPVT include dizziness or fainting associated with exercise or emotional stress. Episodes of ventricular tachycardia may cause the heart to stop beating effectively (cardiac arrest), leading to sudden death in children and young adults without recognized heart abnormalities. Treatments for CPVT, include exercise restriction, the use of beta blockers, and automatic implantable cardioverter defibrillators. Other treatments are surgical sympathectomy and treatment with flecainide. Unfortunately, these treatments are not effective for all patients and are limited by patient compliance, medication side effects, or the risk of adverse events such as fatal electrical storms caused by implantable defibrillators. Accordingly, there is a current unmet need for improved compositions and methods for treatment of CPVT and other cardiac diseases characterized by an abnormal heart rhythm.

Atrial fibrillation (AF) currently affects more than 2 million adults in the United States alone, and is the most common type of sustained cardiac arrhythmia in clinic practice. While treatments for AF exist, there is currently no cure and AF is associated with reduced life expectancy. Moreover, AF contributes to about 158,000 deaths in the U.S. alone, each year.

Accordingly, there is a need for improved compositions and methods for treatment of AF.

### SUMMARY OF THE INVENTION

As described below, the invention of the disclosure features Ca²⁺-calmodulin dependent kinase II (CaMKII) inhibitory multimeric polypeptides (e.g., comprising autocamtide-2-related inhibitory peptide (AIP) multimers), polynucleotides encoding CaMKII inhibitory multimeric polypeptides, and methods of using such polypeptides and polynucleotide for the treatment of a cardiac disease, condition, or disorder characterized by a cardiac arrhythmia (e.g., atrial fibrillation (AF), catecholaminergic polymorphic ventricular tachycardia (CPVT), ischemic heart disease, cardiac arrhythmia, heart failure (e.g., heart failure associated with aortic binding), hypertensive heart disease and pulmonary hypertensive heart disease, myocardial infarction, valvular disease, congenital heart disease, myocardial hypertrophy, ventricular arrhythmia, or Timothy syndrome).

In one aspect, the invention of the disclosure features a multimeric polypeptide that inhibits CaMKII.

In one aspect, the invention of the disclosure features an expression vector comprising a polynucleotide encoding the multimeric polypeptide of the above aspect, or embodiments thereof.

In one aspect, the invention of the disclosure features a pharmaceutical composition comprising an effective amount of the multimeric polypeptide of any of the above aspects, or embodiments thereof.

In one aspect, the invention of the disclosure features a pharmaceutical composition comprising an effective amount of the expression vector of any of the above aspects, or embodiments thereof.

In one aspect, the invention of the disclosure features a cell comprising the expression vector of any of the above aspects, or embodiments thereof.

In one aspect, the invention of the disclosure features a method for modulating a cardiac arrhythmia in a subject. The method involves contacting a cell in the subject comprising a cardiac ryanodine channel (RYR2) with the multimeric polypeptide of any of the above aspects, or embodiments thereof, or a polynucleotide encoding the multimeric polypeptide.

In one aspect, the invention of the disclosure features a method for inhibiting phosphorylation of a ryanodine channel (RYR2) polypeptide in a cell. The method involves contacting a cell comprising a cardiac ryanodine channel (RYR2) with the multimeric polypeptide of any of the above aspects, or embodiments thereof, or a polynucleotide encoding the multimeric polypeptide.

In one aspect, the invention of the disclosure features a method of treating a subject comprising a mutation associated with a cardiac arrhythmia. The method involves administering to the subject the multimeric polypeptide of any of the above aspects, or embodiments thereof, or a polynucleotide encoding the multimeric polypeptide.

In one aspect, the invention of the present disclosure features a method of treating a subject having a cardiac disease, condition, or disorder characterized by a cardiac arrhythmia, comprising administering a multimeric AIP polypeptide or a polynucleotide encoding said polypeptide to the subject.

In one aspect, the invention of the present disclosure features a method of treating a subject having a cardiac disease, condition, or disorder characterized by a cardiac arrhythmia, comprising administering an adeno-associated viral vector comprising a polynucleotide encoding a multimeric AIP polypeptide to the subject.

In one aspect, the invention of the present disclosure features a method of reducing cardiac variability in a subject having atrial fibrillation, comprising administering a multimeric AIP polypeptide or a polynucleotide encoding said multimeric AIP polypeptide to the subject.

In any of the above aspects, or embodiments thereof, the multimeric polypeptide comprises two or more AIP peptides. In any of the above aspects, or embodiments thereof, the multimeric polypeptide comprises from about 3 to about 20 repeats of an AIP peptide. In any of the above aspects, or embodiments thereof, the multimeric polypeptide comprises 3, 4, 5, or 6 repeats of the AIP peptide. In any of the above aspects, or embodiments thereof, the multimeric polypeptide comprises three AIP peptides. In any of the above aspects, or embodiments thereof, the multimeric polypeptide comprises five AIP peptides. In any of the above aspects, or embodiments thereof, the AIP repeats are contiguous and/or separated by linkers. In any of the above aspects, or embodiments thereof, the multimeric polypeptide comprises a sequence having at least 85% amino acid sequence identity to:

(SEQ ID NO: 3)
YKKALHRQEAVDALYKKALHRQEAVDALYKKALHRQEAVDAL
(AIPx3);
or
(SEQ ID NO: 4)
YKKALHRQEAVDALYKKALHRQEAVDALYKKALHRQEAVDALYKKALHR
QEAVDALYKKALHRQEAVDAL (AIPx5)

In any of the above aspects, or embodiments thereof, the multimeric polypeptide is fused to a 12.6-kDa FK506-binding protein (FKBP12.6) polypeptide.

In any of the above aspects, or embodiments thereof, the EC50 for inhibition of CaMKII by the multimeric polypeptide is less than 10% of the EC50 for AIP. In any of the above aspects, or embodiments thereof, the EC50 for the inhibition of CaMKII by the multimeric polypeptide is less than 5% of the EC50 for AIP.

In any of the above aspects, or embodiments thereof, the multimeric polypeptide is operably linked to a promoter suitable for driving expression of the multimeric polypeptide in a mammalian cardiac cell. In any of the above aspects, or embodiments thereof, the promoter is selected from one or more of a cardiac troponin T promoter, an α-myosin heavy chain (α-MHC) promoter, a myosin light chain-2v (MLC-2v) promoter and a cardiac NCX1 promoter.

In any of the above aspects, or embodiments thereof, the vector is a retroviral, adenoviral, or adeno-associated viral vector (AAV). In embodiments, the AAV is selected from one or more of AAV9, AAV6, AAV2i8, AAVrh10, AAVrh74, MyoAAV, Anc80, and Anc82.

In any of the above aspects, or embodiments thereof, the cell is in vivo or in vitro. In any of the above aspects, or embodiments thereof, the cell is a human cell in vivo.

In any of the above aspects, or embodiments thereof, the mutation is in a cardiac ryanodine channel (RYR2). In any of the above aspects, or embodiments thereof, the mutation is RYR2^{R4651I}.

In any of the above aspects, or embodiments thereof, the method inhibits a cardiac arrhythmia. In any of the above aspects, or embodiments thereof, the arrhythmia is catecholaminergic polymorphic ventricular tachycardia. In any of the above aspects, or embodiments thereof, the arrhythmia is atrial fibrillation.

In any of the above aspects, or embodiments thereof, the subject is a mammal and/or the cell is from a mammal. In any of the above aspects, or embodiments thereof, the mammal is a human.

In any of the above aspects, or embodiments thereof, the polynucleotide is DNA, RNA, or a combination thereof. In any of the above aspects, or embodiments thereof, the polynucleotide comprises one or more modified nucleobases. In any of the above aspects, or embodiments thereof, the polynucleotide is present in a vector.

In any of the above aspects, or embodiments thereof, the adeno-associated viral vector is an effective amount of an adeno-associated viral vector. In any of the above aspects, or embodiments thereof, the effective amount is between about 1×10¹⁰ viral genomes/kg and about 1×10¹⁴ viral genomes/kg. In any of the above aspects, or embodiments thereof, the effective amount is effective to transfect between about 20% and about 40% of myocytes in the subject.

In any of the above aspects, or embodiments thereof, the administering is effective to reduce heart rate variability in the subject. In any of the above aspects, or embodiments thereof, the administering is effective to reduce atrial scarring in the subject.

In any of the above aspects, or embodiments thereof, the cardiac disease, condition, or disorder is atrial fibrillation.

In any of the above aspects, or embodiments thereof, the multimeric AIP comprises between about 3-10 repeats of AIP.

Compositions and articles defined by the invention were isolated or otherwise manufactured in connection with the examples provided below. Other features and advantages of the invention will be apparent from the detailed description, and from the claims.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “multimer” or “multimeric polypeptide” is meant a polypeptide sequence containing two or more repeats of an amino acid sequence. In one embodiment, the multimeric polypeptide inhibits CaMKII. Such multimeric polypeptides are termed “CaMKII inhibitory multimeric polypeptides.”

By “Autocamtide-2-related inhibitory peptide (AIP)” is meant a peptide or fragment thereof having at least about 85% amino acid sequence identity to YKKALRRQEAVDAL (SEQ ID NO: 1); comprising or consisting of at least about 9-14 contiguous amino acids of SEQ ID NO: 1; and having cardiac regulatory activity and/or CaMKII inhibitory activity. In an embodiment, an AIP has at least about 85% amino acid sequence identity to KKALRRQEAVDAL(SEQ ID NO: 5). In some instances, an AIP has at least about 85% amino acid sequence identity to kkKlrrgeaFdal (AIPo) (SEQ ID NO: 6), where capital letters indicate alterations to the amino acid sequence KKALRRQEAVDAL (SEQ ID NO: 5). In some cases, an AIP contains one or more of the alterations described in Ishida, et al, “Critical amino acid residues of AIP, a highly specific inhibitory peptide of calmodulin-dependent protein kinase II,” FEBSLetters, 427:115-118 (1998), the disclosure of which is incorporated herein by reference in its entirety for all purposes, and/or the AIP contains an alteration at one or more of the critical amino acid residues described therein (e.g., at the sites corresponding to the capitalized amino acid residues provided in the amino acid sequence kkKlrrgeaFdal (SEQ ID NO: 6)). In some instances an AIP has at least 70%, 75%, 80%, 85%, or 90% amino acid sequence identity to KKALRRQEAVDAL (SEQ ID NO: 5) and contains one or more of the following amino acids substitutions or combinations thereof: K1A, K1Y, K2A, K2Y, A3K, A3Y, L4A, L4F, L4N1e (Noroleucine), L4G, L4I, LrNva (Norvaline), L4M, L4V, L4Y, R5A, R5K, R5H, R5Y, R6A, R6Orn, R6K, R6dmR (N^{G},N^{G}-dimethyl-arginine), R6Cit (Citruline), R6H, R6Y, Q7A, Q7E, Q7D, Q7N, Q70m, Q7Y, E8A, E8Y, A9G, A9C, A9V, A9I, A9L, A9Y, V10A, V10F, V10I, V10L, V10Nva (Norvaline), V10Abu (2-Aminobutylic acid), V10G, V10Y, D11A, D11Y, A12Y, L13A, L13Y, and A3K/V10F. In embodiments, AIPo has increased selectivity for CaMKII inhibition vs inhibition of PKC. In one embodiment, the AIP peptide comprises one or more alterations in the peptide sequence. In one embodiment, the AIP peptide consists essentially of SEQ. ID. NO: 1. In another embodiment, the AIP peptide consists of SEQ. ID. NO: 1 or consists of about 9-13 contiguous amino acids of SEQ. ID. NO: 1. In one embodiment, the AIP peptide consists essentially of SEQ. ID. NO: 1 or consists essentially of about 9-13 contiguous amino acids of SEQ. ID. NO: 1. In another embodiment, the AIP peptide comprises one or more modified amino acids. In another embodiment, the AIP peptide comprises 1, 2, 3, 4, 5 or more alterations in SEQ ID NO:1.

By “Autocamtide-2-related inhibitory (AIP) multimeric polypeptide” is meant a polypeptide comprising 2 or more repeats of an AIP peptide. In one embodiment, the AIP multimer comprises between 2 and 20 repeats (i.e., 1, 2, 3, 4, 5, 6, 7, 8, 9, 10, 11, 12, 13, 14, 16, 17, 18, 19, 20). In one embodiment, the AIP multimeric polypeptide contains the sequence YKKALHRQEAVDALYKKALHRQEAVDALYKKALHRQEAVDAL (SEQ ID NO: 3) or the sequence YKKALHRQEAVDALYKKALHRQEAVDALYKKALHRQEAVDALYKKALHRQEAVDALYKKALHRQ EAVDAL (SEQ ID NO: 4). In another embodiment, the repeats are contiguous.

By “AIP multimeric polynucleotide” is meant a polynucleotide that encodes an AIP multimer polypeptide.

By “calcium/calmodulin-dependent protein kinase II (CaMKII) polypeptide” is meant a polypeptide or fragment thereof having at least 85% sequence identity to the amino acid sequence of GenBank Accession No. AAH32784.1, which is provided below, or a fragment thereof that has kinase activity.

>AAH32784.1 Calcium/calmodulin-dependent protein
kinase II delta [Homo sapiens]
(SEQ ID NO: 7)
MASTTTCTRFTDEYQLFEELGKGAFSVVRRCMKIPTGQEYAAKIINTKK
LSARDHQKLEREARICRLLKHPNIVRLHDSISEEGFHYLVFDLVTGGEL
FEDIVAREYYSEADASHCIQQILESVNHCHLNGIVHRDLKPENLLLASK
SKGAAVKLADFGLAIEVQGDQQAWFGFAGTPGYLSPEVLRKDPYGKPVD
MWACGVILYILLVGYPPFWDEDQHRLYQQIKAGAYDFPSPEWDTVTPEA
KDLINKMLTINPAKRITASEALKHPWICQRSTVASMMHRQETVDCLKKF
NARRKLKGAILTTMLATRNFSAAKSLLKKPDGVKESTESSNTTIEDEDV
KARKQEIIKVTEQLIEAINNGDFEAYTKICDPGLTAFEPEALGNLVEGM
DFHRFYFENALSKSNKPIHTIILNPHVHLVGDDAACIAYIRLTQYMDGS
GMPKTMQSEETRVWHRRDGKWQNVHFHRSGSPTVPIK.

By “calcium/calmodulin-dependent protein kinase II (CaMKII) polynucleotide” is meant a polynucleotide that encodes a CaMKII polypeptide. A representative CaMKII polynucleotide sequence is provided below (GenBank Accession No. BC032784.1).

>BC032784.1:63-1499 Homo sapiens calcium/
calmodulin-dependent protein kinase II delta, mRNA
(cDNA clone MGC:44911 IMAGE:5178265), complete cds
(SEQ ID NO: 8)
ATGGCTTCGACCACAACCTGCACCAGGTTCACGGACGAGTATCAGCTTTT
CGAGGAGCTTGGAAAGGGGGCATTCTCAGTGGTGAGAAGATGTATGAAAA
TTCCTACTGGACAAGAATATGCTGCCAAAATTATCAACACCAAAAAGCTT
TCTGCTAGGGATCATCAGAAACTAGAAAGAGAAGCTAGAATCTGCCGTCT
TTTGAAGCACCCTAATATTGTGCGACTTCATGATAGCATATCAGAAGAGG
GCTTTCACTACTTGGTGTTTGATTTAGTTACTGGAGGTGAACTGTTTGAA
GACATAGTGGCAAGAGAATACTACAGTGAAGCTGATGCCAGTCATTGTAT
ACAGCAGATTCTAGAAAGTGTTAATCATTGTCACCTAAATGGCATAGTTC
ACAGGGACCTGAAGCCTGAGAATTTGCTTTTAGCTAGCAAATCCAAGGGA
GCAGCTGTGAAATTGGCAGACTTTGGCTTAGCCATAGAAGTTCAAGGGGA
CCAGCAGGCGTGGTTTGGTTTTGCTGGCACACCTGGATATCTTTCTCCAG
AAGTTTTACGTAAAGATCCTTATGGAAAGCCAGTGGATATGTGGGCATGT
GGTGTCATTCTCTATATTCTACTTGTGGGGTATCCACCCTTCTGGGATGA
AGACCAACACAGACTCTATCAGCAGATCAAGGCTGGAGCTTATGATTTTC
CATCACCAGAATGGGACACGGTGACTCCTGAAGCCAAAGACCTCATCAAT
AAAATGCTTACTATCAACCCTGCCAAACGCATCACAGCCTCAGAGGCACT
GAAGCACCCATGGATCTGTCAACGTTCTACTGTTGCTTCCATGATGCACA
GACAGGAGACTGTAGACTGCTTGAAGAAATTTAATGCTAGAAGAAAACTA
AAGGGTGCCATCTTGACAACTATGCTGGCTACAAGGAATTTCTCAGCAGC
CAAGAGTTTGTTGAAGAAACCAGATGGAGTAAAGGAGTCAACTGAGAGTT
CAAATACAACAATTGAGGATGAAGATGTGAAAGCACGAAAGCAAGAGATT
ATCAAAGTCACTGAACAACTGATCGAAGCTATCAACAATGGGGACTTTGA
AGCCTACACAAAAATCTGTGACCCAGGCCTTACTGCTTTTGAACCTGAAG
CTTTGGGTAATTTAGTGGAAGGGATGGATTTTCACCGATTCTACTTTGAA
AATGCTTTGTCCAAAAGCAATAAACCAATCCACACTATTATTCTAAACCC
TCATGTACATCTGGTAGGGGATGATGCCGCCTGCATAGCATATATTAGGC
TCACACAGTACATGGATGGCAGTGGAATGCCAAAGACAATGCAGTCAGAA
GAGACTCGTGTGTGGCACCGCCGGGATGGAAAGTGGCAGAATGTTCATTT
TCATCGCTCGGGGTCACCAACAGTACCCATCAAGTAA.

By “CN19o peptide” is meant a peptide or fragment thereof having at least about 85% amino acid sequence identity to KRAPKLGQIGRQKAVDIED (SEQ ID NO:2); comprising or consisting of at least about 9-19 contiguous amino acids of SEQ ID NO:2; and having cardiac regulatory activity and/or CaMKII inhibitory activity. In one embodiment, the CN19o peptide comprises one or more alterations in the peptide sequence.

By “CN19o multimeric polypeptide” is meant a polypeptide or fragment thereof comprising 2 or more repeats of a CN19o peptide. In one embodiment, the CN19o multimer comprises the sequence KRAPKLGQIGRQKAVDIEDKRAPKLGQIGRQKAVDIEDKRAPKLGQIGRQKAVDIED (SEQ ID NO: 9) or the sequence KRAPKLGQIGRQKAVDIEDKRAPKLGQIGRQKAVDIEDKRAPKLGQIGRQKAVDIEDKRAPKLG QIGRQKAVDIEDKRAPKLGQIGRQKAVDIED (SEQ ID NO: 10). In another embodiment, the repeats are contiguous.

By “CN19o multimeric polynucleotide” is meant a polynucleotide that encodes a CN19o multimer polypeptide.

By “12.6-kDa FK506-binding protein (FKBP12.6) polypeptide” is meant a polypeptide or fragment thereof having at least 85% sequence identity to the amino acid sequence of NCBI Ref. Seq. Accession No. NP_004107.1, which is provided below, or a fragment thereof that is capable of binding an RYR2 polypeptide.

>NP_004107.1 peptidyl-prolyl cis-trans isomerase
FKBPIB isoform a [Homo sapiens]
(SEQ ID NO: 11)
MGVEIETISPGDGRTFPKKGQTCVVHYTGMLQNGKKFDSSRDRNKPFKF
RIGKQEVIKGFEEGAAQMSLGQRAKLTCTPDVAYGATGHPGVIPPNATL
IFDVELLNLE.

By “12.6-kDa FK506-binding protein (FKBP12.6) polynucleotide” is meant a polynucleotide that encodes an FKBP12.6 polypeptide. A representative FKBP12.6 polynucleotide sequence is provided below (NCBI Ref. Seq. Accession No. NM_004116.5).

>NM 004116.5:110-436 Homo sapiens FKBP prolyl
isomerase 1B (FKBP1B), transcript variant 1, mRNA
(SEQ ID NO: 12)
ATGGGCGTGGAGATCGAGACCATCTCCCCCGGAGACGGAAGGACATTCC
CCAAGAAGGGCCAAACGTGTGTGGTGCACTACACAGGAATGCTCCAAAA
TGGGAAGAAGTTTGATTCATCCAGAGACAGAAACAAACCTTTCAAGTTC
AGAATTGGCAAACAGGAAGTCATCAAAGGTTTTGAAGAGGGTGCAGCCC
AGATGAGCTTGGGGCAGAGGGCGAAGCTGACCTGCACCCCTGATGTGGC
ATATGGAGCCACGGGCCACCCCGGTGTCATCCCTCCCAATGCCACCCTC
ATCTTTGACGTGGAGCTGCTCAACTTAGAGTGA.

By “ryanodine receptor 2 (RYR2) polypeptide” is meant a polypeptide or fragment thereof having at least 85% sequence identity to the amino acid sequence of GenBank Accession No. CAA62975.1, which is provided below, or a fragment thereof capable of forming a homotetramer that functions as a calcium channel.

>CAA62975.1 ryanodine receptor, partial
[Homo sapiens]
(SEO ID NO: 13)
LYELLAALIRGNRKNCAQFSGSLDWLISRLERLEASSGILEVLHCVLVE
SPEALNIIKEGHIKSIISLLDKHGRNHKVLDVLCSLCVCHGVAVRSNQH
LICDNLLPGRDLLLQTRLVNHVSSMRPNIFLGVSEGSAQYKKWYYELMV
DH.

By “ryanodine receptor 2 (RYR2) polynucleotide” is meant a polynucleotide that encodes a RYR2 polypeptide. A representative RYR2 polynucleotide sequence is provided below (GenBank Accession No. X91869.1).

>X91869.1 H. sapiens mRNA for ryanodine receptor
(SEQ ID NO: 14)
CTGTATGAGTTGCTGGCGGCTCTAATTAGAGGAAATCGTAAAAACTGTG
CTCAATTTTCTGGCTCCCTCGACTGGTTGATCAGCAGATTGGAAAGACT
GGAAGCTTCTTCAGGCATTCTGGAAGTTTTACACTGTGTTTTAGTAGAA
AGTCCAGAAGCTCTAAATATTATTAAAGAAGGACATATTAAATCTATTA
TCTCACTTTTAGACAAACATGGAAGAAATCACAAGGTTCTGGATGTCTT
GTGCTCACTCTGTGTTTGCCACGGGGTTGCAGTCCGTTCTAACCAGCAT
CTCATCTGTGACAATCTCCTACCAGGAAGAGACTTGTTATTGCAGACAC
GTCTTGTGAACCATGTCAGCAGCATGAGACCCAATATTTTTCTGGGCGT
CAGTGAAGGTTCTGCTCAGTATAAGAAATGGTACTATGAATTGATGGTG
GACCAC.

By “CaMKII inhibitor” is meant a peptide or small molecule that inhibits the activity of CaMKII. Exemplary inhibitors are known in the art (e.g., AIP, CN19, CN27, CN19o, CN21) and described, for example, by Coultrap et al., PLOS One e25245, Vol 6, Issue 10, 2011 and Pellicena et al., Frontiers in Pharmacology 21:1-20, 2014. Other inhibitors include the following:

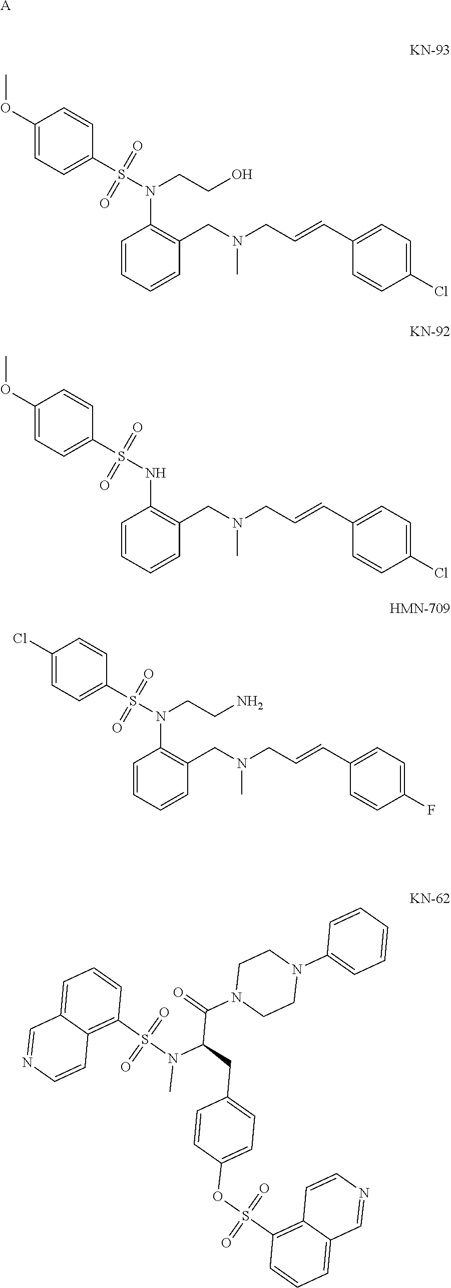

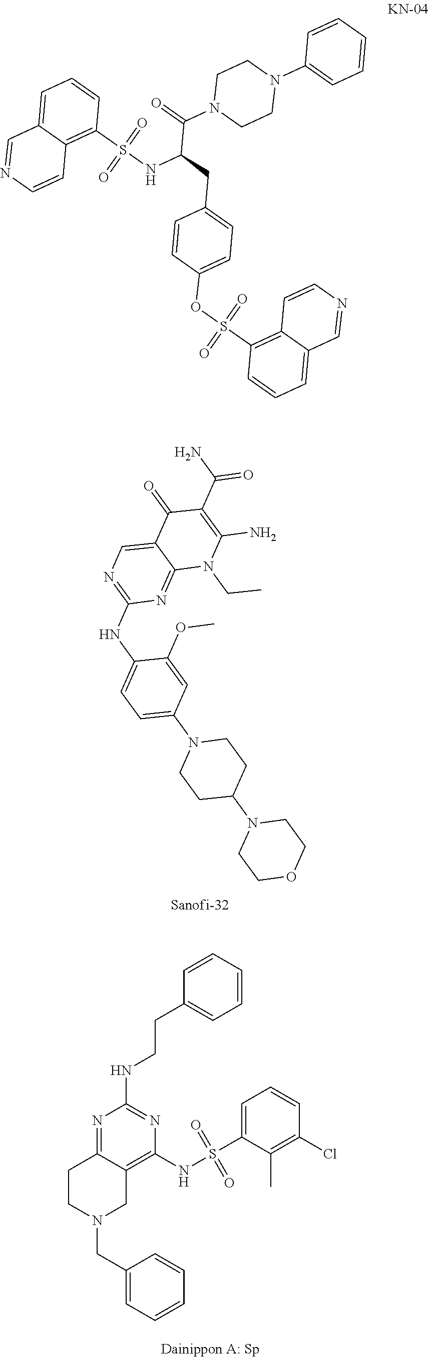 [SMILES: O=C([C@@H](Cc1ccc(OS(=O)(=O)c2cccc3cnccc23)cc1)NS(=O)(=O)c1cccc2cnccc12)N1CCN(c2ccccc2)CC1 ; CCn1c(N)c(C(N)=O)c(=O)c2cnc(Nc3ccc(N4CCC(N5CCOCC5)CC4)cc3OC)nc21 ; Cc1c(Cl)cccc1S(=O)(=O)Nc1nc(NCCc2ccccc2)nc2c1CN(Cc1ccccc1)CC2]

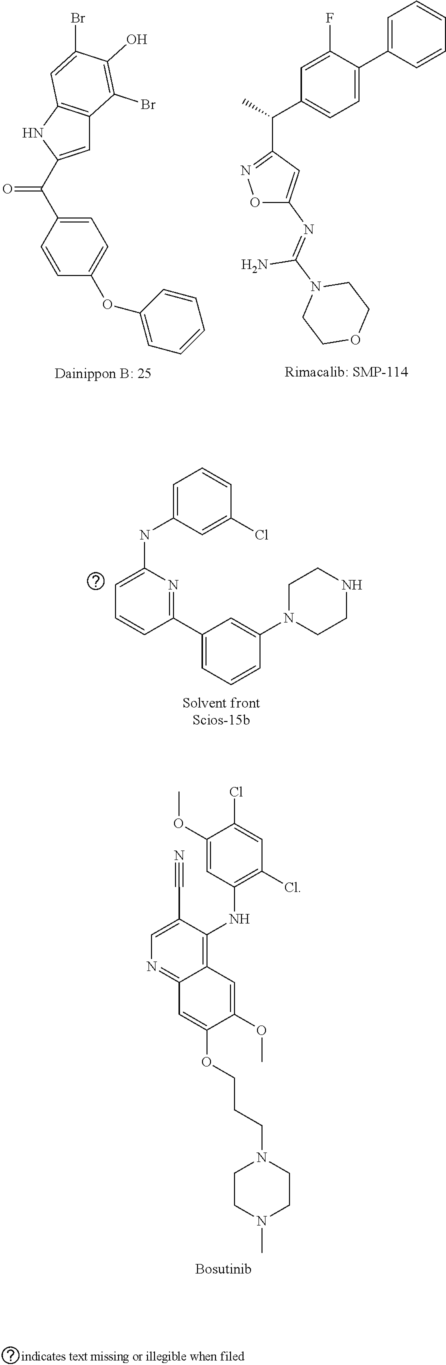 [SMILES: O=C(c1ccc(Oc2ccccc2)cc1)c1cc2c(Br)c(O)c(Br)cc2[nH]1 ; C[C@H](c1ccc(-c2ccccc2)c(F)c1)c1cc(/N=C(\N)N2CCOCC2)on1 ; Clc1cccc(Nc2cccc(-c3cccc(N4CCNCC4)c3)n2)c1 ; COc1cc(Nc2c(C#N)cnc3cc(OCCCN4CCN(C)CC4)c(OC)cc23)c(Cl)cc1Cl]

By “agent” is meant a peptide, polypeptide, nucleic acid molecule, or small compound.

By “ameliorate” is meant decrease, suppress, attenuate, diminish, arrest, or stabilize the development or progression of a disease. In some embodiments, the disease is a cardiac disease or disorder.

By “alteration” with reference to an amino acid sequence means a change in the identity of one or more amino acids of the amino acid sequence.

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid. A polynucleotide analog retains the biological activity of a corresponding naturally-occurring polypeptide, while having certain biochemical modifications that enhance the analog's function relative to a naturally occurring polynucleotide. Such biochemical modifications could increase the analog's nuclease resistance, membrane permeability, or half-life, without altering, for example, functional activity, such as its protein encoding function. An analog may include a modified nucleic acid molecule.

The term “cardiomyocyte” as used herein broadly refers to a muscle cell of the heart. In one embodiment, a mammalian cardiac cell is a cardiomyocyte. In another embodiment, a cardiomyocyte that is differentiated from an induced pluripotent stem cell is a cardiomyocyte.

As used herein, the phrase “cardiac condition, disease, or disorder” is intended to include all disorders characterized by insufficient, undesired or abnormal cardiac function. Exemplary cardiac conditions, diseases or disorders include, but are not limited to, atrial fibrillation (AF), catecholaminergic polymorphic ventricular tachycardia (CPVT), ischemic heart disease, cardiac arrhythmia, heart failure (e.g., heart failure associated with aortic binding), hypertensive heart disease and pulmonary hypertensive heart disease, myocardial infarction, valvular disease, congenital heart disease, myocardial hypertrophy, ventricular arrhythmia, or Timothy syndrome and any condition which leads to congestive heart failure in a subject, particularly a human subject. Insufficient or abnormal cardiac function can be the result of disease, injury, genetic mutations, and/or aging. By way of background, a response to myocardial injury follows a well-defined path in which some cells die while others enter a state of hibernation where they are not yet dead but are dysfunctional. This is followed by infiltration of inflammatory cells, deposition of collagen as part of scarring, all of which happen in parallel with in-growth of new blood vessels and a degree of continued cell death.

By “effective amount” or “therapeutically effective amount” is meant the amount of an agent required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount. The term “therapeutically effective amount” therefore refers to an amount of the composition as disclosed herein that is sufficient to, for example, effect a therapeutically or prophylactically significant reduction in a symptom or clinical marker associated with a cardiac dysfunction or disorder when administered to a typical subject who has a cardiovascular condition, disease or disorder.

With reference to the treatment of, for example, a cardiovascular condition or disease in a subject, the term “therapeutically effective amount” refers to an amount that is safe and sufficient to prevent or delay the development or a cardiovascular disease or disorder (e.g., cardiac arrhythmia). The amount can thus cure or cause the arrhythmia to be suppressed, or to cause the cardiovascular disease or disorder to go into remission, slow the course of cardiovascular disease progression, slow or inhibit a symptom of a cardiovascular disease or disorder, slow or inhibit the establishment of secondary symptoms of a cardiovascular disease or disorder or inhibit the development of a secondary symptom of a cardiovascular disease or disorder. The effective amount for the treatment of the cardiovascular disease or disorder depends on the type of cardiovascular disease to be treated, the severity of the symptoms, the subject being treated, the age and general condition of the subject, the mode of administration and so forth. Thus, it is not possible to specify the exact “effective amount”. However, for any given case, an appropriate “effective amount” can be determined by one of ordinary skill in the art using only routine experimentation. The efficacy of treatment can be judged by an ordinarily skilled practitioner, for example, efficacy can be assessed in animal models of a cardiovascular disease or disorder as discussed herein, for example treatment of a rodent with acute myocardial infarction or ischemia-reperfusion injury, and any treatment or administration of the compositions or formulations that leads to a decrease of at least one symptom of the cardiovascular disease or disorder as disclosed herein, for example, increased heart ejection fraction, decreased rate of heart failure, decreased infarct size, decreased associated morbidity (pulmonary edema, renal failure, arrhythmias) improved exercise tolerance or other quality of life measures, and decreased mortality indicates effective treatment. In embodiments where the compositions are used for the treatment of a cardiovascular disease or disorder, the efficacy of the composition can be judged using an experimental animal model of cardiovascular disease, e.g., animal models of ischemia-reperfusion injury (Headrick J P, Am J Physiol Heart circ Physiol 285; H1797; 2003) and animal models acute myocardial infarction. (Yang Z, Am J Physiol Heart Circ. Physiol 282:H949: 2002; Guo Y, J Mol Cell Cardiol 33; 825-830, 2001). When using an experimental animal model, efficacy of treatment is evidenced when a reduction in a symptom of the cardiovascular disease or disorder, for example, a reduction in one or more symptom of dyspnea, chest pain, palpitations, dizziness, syncope, edema, cyanosis, pallor, fatigue and high blood pressure which occurs earlier in treated, versus untreated animals.

Subjects amenable to treatment by the methods as disclosed herein can be identified by any method to diagnose cardiac arrhythmia. Methods of diagnosing these conditions are well known by persons of ordinary skill in the art. By way of non-limiting example, cardiac arrhythmia can be diagnosed by electrocardiogram (ECG or EKG) which is a graphic recordation of cardiac activity, either on paper or a computer monitor.

The terms “coronary artery disease” and “acute coronary syndrome” as used interchangeably herein, and refer to myocardial infarction refer to a cardiovascular condition, disease or disorder, include all disorders characterized by insufficient, undesired or abnormal cardiac function, e.g. ischemic heart disease, hypertensive heart disease and pulmonary hypertensive heart disease, valvular disease, congenital heart disease and any condition which leads to congestive heart failure in a subject, particularly a human subject. Insufficient or abnormal cardiac function can be the result of disease, injury and/or aging. By way of background, a response to myocardial injury follows a well-defined path in which some cells die while others enter a state of hibernation where they are not yet dead but are dysfunctional. This is followed by infiltration of inflammatory cells, deposition of collagen as part of scarring, all of which happen in parallel with in-growth of new blood vessels and a degree of continued cell death.

In this disclosure, “comprises,” “comprising,” “containing” and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments. Any embodiments specified as “comprising” a particular component(s) or element(s) are also contemplated as “consisting of” or “consisting essentially of” the particular component(s) or element(s) in some embodiments.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 5, 6, 7, 8, 9, 10, 11, 12, 13, 14, 15, 20, 25, 30, 40, 45, 50, 60, 70, 80, 90, 100, 200, 300 400, 500, or 1000 nucleotides or amino acids.

By “heart rate variability” or “beat-to-beat variability” is meant the variability in the RR interval (the interval between two successive R-waves as measured by an electrocardiogram), particularly as measured by the standard deviation of the RR interval. In some embodiments, an increase in heart rate variability or beat-to-beat variability, in a subject, relative to a reference, is indicative of an arrhythmia or is a symptom of cardiovascular disease, condition, or disorder. In some embodiments, administering agents disclosed herein to a subject having an increase in heart rate variability or beat-to-beat variability, relative to a reference, is effective to decrease heart rate variability or beat-to-beat variability, preferably where such administration is effective to decrease heart rate variability or beat-to-beat variability to reference levels.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

As used herein, “obtaining” as in “obtaining an agent” includes synthesizing, purchasing, or otherwise acquiring the agent.

By “polypeptide” or “amino acid sequence” is meant any chain of amino acids, regardless of length or post-translational modification. In various embodiments, the post-translational modification is glycosylation or phosphorylation. In various embodiments, conservative amino acid substitutions may be made to a polypeptide to provide functionally equivalent variants, or homologs of the polypeptide. In some aspects the invention embraces sequence alterations that result in conservative amino acid substitutions. In some embodiments, a “conservative amino acid substitution” refers to an amino acid substitution that does not alter the relative charge or size characteristics of the protein in which the conservative amino acid substitution is made. Variants can be prepared according to methods for altering polypeptide sequence known to one of ordinary skill in the art such as are found in references that compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Non-limiting examples of conservative substitutions of amino acids include substitutions made among amino acids within the following groups: (a) M, I, L, V; (b) F, Y, W; (c) K, R, H; (d) A, G; (e) S, T; (f) Q, N; and (g) E, D. In various embodiments, conservative amino acid substitutions can be made to the amino acid sequence of the proteins and polypeptides disclosed herein.

By “reduces” is meant a negative alteration relative to a reference. In the context of a cardiac arrhythmia, the term reduces means any significant reduction in symptoms. For example, a reduction of at least 1%, at least 5%, at least 10%, at least 25%, at least 50%, at least 75%, or at least 100% of incidences of arrhythmia or symptoms, or severity of symptoms, including whole integer percentages from 1% to 100%.

By “reference” is meant a corresponding control condition. In one embodiment, the reference is an untreated control. In another embodiment, the reference is a wild-type healthy control. In another embodiment, a subject having a cardiac disease or disorder is treated with a multimer polypeptide described herein and the effect of such treatment is assessed relative to the subject prior to treatment or to an untreated subject also having the cardiac disease or disorder

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 85% identity to a reference amino acid sequence or nucleic acid sequence. In embodiments, such a sequence is at least 90%, 95%, or even 99% identical at the amino acid level or nucleic acid to a reference sequence.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

As used herein, the term “modulate” refers to regulate or adjust to a certain degree.

As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal without the production of undesirable physiological effects such as nausea, dizziness, gastric upset and the like. A pharmaceutically acceptable carrier will not promote the raising of an immune response to an agent with which it is admixed, unless so desired. The preparation of a pharmacological composition that contains active ingredients dissolved or dispersed therein is well understood in the art and need not be limited based on formulation. Typically, such compositions are prepared as injectable either as liquid solutions or suspensions, however, solid forms suitable for solution, or suspensions, in liquid prior to use can also be prepared. The preparation can also be emulsified or presented as a liposome composition. The active ingredient can be mixed with excipients which are pharmaceutically acceptable and compatible with the active ingredient and in amounts suitable for use in the therapeutic methods described herein. Suitable excipients include, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient. The therapeutic composition of the present invention can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active agent used in the methods described herein that will be effective in the treatment of a particular disorder or condition will depend on the nature of the disorder or condition, and can be determined by standard clinical techniques. Suitable pharmaceutical carriers are described in Remington's Pharmaceutical Sciences, A. Osol, a standard reference text in this field of art. For example, a parenteral composition suitable for administration by injection is prepared by dissolving 1.5% by weight of active ingredient in 0.9% sodium chloride solution.

In one embodiment, the “pharmaceutically acceptable” carrier does not include in vitro cell culture media.

In one embodiment, the term “pharmaceutically acceptable” means approved by a regulatory agency of the Federal or a state government or listed in the U.S. Pharmacopeia or other generally recognized pharmacopeia for use in animals, and more particularly in humans. Specifically, it refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations, and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in Remington's Pharmaceutical Sciences, 18th Ed., Gennaro, ed. (Mack Publishing Co., 1990). The formulation should suit the mode of administration.

By “subject” is meant a mammal, including, but not limited to, a human or non-human mammal, such as a bovine, equine, canine, ovine, rodent, or feline. In one embodiment, the subject has or has a propensity to develop a monogenic disease, disorder, or condition that can be treated with the gene therapy vectors, cell-based therapeutics, and methods disclosed elsewhere herein.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The term “tissue” refers to a group or layer of similarly specialized cells which together perform certain special functions. The term “tissue-specific” refers to a source or defining characteristic of cells from a specific tissue.

As used herein, the terms “treat,” treating,” “treatment,” and the like refer to reducing or ameliorating a disorder and/or symptom associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIGS. 1A-1C provide a schematic, plots, and a bar graph showing how the peptide inhibitor autocamtide-2-related inhibitory peptide (AIP) can be used as part of a gene therapy for catecholaminergic polymorphic ventricular tachycardia (CPVT). FIG. 1A provides a schematic showing how the AIP can be delivered to a cell (e.g. a mouse cell) using a clinical vector (e.g. adeno-associated virus (AAV)) to inhibit or prevent phosphorylation by CaMKII of CPVT mutant RYR2 (R4650I in a mouse polypeptide is shown as one example). In addition to the inhibitory peptide cargo, the clinical vector contains a promoter and a capsid, either of which may be optimized to increase efficacy of the vector in treatment. In FIG. 1A, RYR2 represents “cardiac ryanodine receptor 2.” FIG. 1B provides an electrocardiogram for a mouse treated with AAV that expresses either a negative control (green fluorescent protein (GFP)) or AIP. The mouse treated with AIP showed reduced ventricular tachycardia (VT) following pacing, compared to the mouse treated with GFP. FIG. 1C provides a bar graph showing percent of mice showing induced ventricular tachycardia (VT) in wild-type mice or RYR2-R176Q+/1 mice (CPVT mice with the RyR2 R176Q mutation) treated with AIP or GFP. The fractions in the bars represent the number of mice showing induced VT over the total number of studied mice. Administration of AIP resulted in a reduction in induced VT in the RYR2-R176Q+/− mice.

FIGS. 2A-2G provide plots, a protein structure, schematics, a bar graph, and images showing how a therapeutic cargo containing a CaMKII inhibitor was optimized to treat catecholaminergic polymorphic ventricular tachycardia (CPVT) through altering potency, binding, and/or multimerization of the CaMKII inhibitor. FIG. 2A provides plots showing the IC50 concentrations for the autocamtide-2-related inhibitory peptide (AIP) and CN19o (Coultrap, et al. “Improving a Natural CaMKII Inhibitor by Random and Rational Design,” PLoS One, Oct. 3, 2011, doi.org/10.1371/journal.pone.0025245; and Ishida, et al, “Critical amino acid residues of AIP, a highly specific inhibitory peptide of calmodulin-dependent protein kinase II,” FEBS Letters, 427:115-118 (1998), the disclosures of which is incorporated herein by reference in its entirety for all purposes). CN19o has a lower IC50 than AIP and is, therefore, a more potent inhibitor of CaMKII activity in the in vitro system used for these assays. FIG. 2B provides an EM map for RYR2 at 6 Angstrom resolution. In FIG. 2B, FKBP12.6 represents the “FK506 binding protein” that stabilizes RyR2 preventing aberrant activation of the channel during the resting phase of the cardiac cycle. FKBP12.6 stabilizes RyR2 in a closed state. FKBP12.6 serves as a stabilizer to enhance rigidity of the HD2 (HD2 and HD2′) and P2 (P2 and P2′) domains (Chi, et al. “Molecular basis for allosteric regulation of the type 2 ryanodine receptor channel gating by key modulators,” PNAS, 116:25575-25582 (2019), the disclosure of which is incorporated herein by reference in its entirety for all purposes). FIG. 2C provides a schematic showing an AIP×5 multimer. FIG. 2D provides a schematic showing the design of an experiment to evaluate the effectiveness of different candidate therapeutic cargos for reducing arrhythmia in a CPVT mouse model (RYR2-R46450I). The left portion of FIG. 2D provides a schematic representation of different polypeptides that may be contained within the therapeutic cargo to be delivered to the subject. In FIG. 2D, P2A represents a self-cleaving peptide, AIP represents an instance of the autocamtide-2-related inhibitory peptide (AIP), mCherry represents the fluorescent protein mCherry, CN19o represents the CaMKII inhibitor CN19o, FKBP12.6 represents a FKBP12.6 polypeptide that binds to RYR2, and the polygonal shape represents a viral capsid. The rightmost portion of FIG. 2D (FIG. 2D (CONTINUED)) shows electrocardiograms for WT mice and RYR2^{R4650/WT} mice. The RYR2^{R4650/WT} mice show irregularities in their heartbeat. FIG. 2E provides a plot showing percent ectopy in RYR2^{R4650I/WT} mice administered the indicated polypeptides described in FIG. 2D. Administration of AIP×5 resulted in the greatest reduction in instances of ectopy observed in the mice. FIG. 2F provides a bar graph showing the percent of RYR2^{R4650I/WT} mice administered the indicated polypeptides described in FIG. 2D that showed induced ventricular tachycardia (VT). Administration of AIP×5 was associated with a reduction in instances of induced VT in the mice. FIG. 2G provides images of cardiac tissue showing mCherry expression in the tissue and, thereby, confirming that the constructs described in FIG. 2D were delivered to and expressed in cardiac tissue of the RYR2^{R4650I/WT} mice.

FIG. 3 provides a chart, a capillary Western image, and a plot showing the effects of the indicated constructs, which are described in FIG. 2D, on CaMKII activity in vivo. To confirm suppression of CaMKII activation in vivo, phosphorylation of phospholamban (PLB) at threonine-17, a known target of CaMKII, was analyzed from whole heart lysates. The middle panel of FIG. 3 provides a capillary Western image showing glyceraldehyde-3-phosphate dehydrogenase polypeptide (GAPDH) expression in cells contacted (+) or not contacted (−) with isoproterenol (ISO), as well as the levels of phosphorylated PLB (pPLB) and non-phosphorylated PLB (PLB). The right panel of FIG. 3 provides a plot showing the ratio of pPLB to PLB in hearts treated with no ISO or with ISO and mCherry (the “ISO” sample), AIP, AIP×5, AIP-FKBP12.6, CN19o, CN19oX3, or CN19o-FKBP12.6, which are described in FIG. 2D.

FIG. 4 provides a schematic illustrating the design of an in vitro experiment to evaluate the impact of the purified polypeptides described in FIG. 2D and shown in the left portion of FIG. 4 on CaMKIIδ (an isomer of CaMKII) activity. The sequences of the polypeptides used in the in vitro experiment are provided in Table 1. Activity of CaMKIIδ was measured using a bioluminescent and homogeneous ADP monitoring assay for kinases (ADP-GLO™), which is described in Zegzouti, et al. “ADP-Glo: A Bioluminescent and Homogeneous ADP Monitoring Assay for Kinases,” ASSAY and Drug Development Technologies, December 2009, 560-572, doi.org/10.1089/adt.2009.0222, the disclosure of which is incorporated herein by reference in its entirety for all purposes.

FIG. 5 provides a plot from an in vitro bioluminescent and homogeneous ADP monitoring assay showing that AIP multimerization improved potency of CaMKII inhibition (i.e., a reduction in EC50) while CN19o multimerization did not improve potency of CaMKII inhibition. Multimerization of CN19o led to a decrease in CaMKII inhibition (i.e., an increase in EC50). The sequences for the peptides are provided in Table 1.

FIG. 6 provides a schematic showing administration of AAV vectors to RYR2^{R176Q/WT} and RYR2^{R4560I/WT} mice. RYR2^{R176Q/WT} and RYR2^{R4560I/WT} mice. The top portion of FIG. 6 provides a schematic showing the components of polynucleotides of the AAV vectors. In FIG. 6, ITR represents “inverted terminal repeat,” CASQ2 represents a calsequestrin 2 enhancer, cTnT represents a “cardiac troponin” promoter, an INT represents a hemoglobin enhancer, AIP×5 represents a multimer containing five autocamtide-2-related inhibitory peptide (AIP) units, mScarlet represents the fluorescent polypeptide mScarlet, nls represents a nuclear localization signal, “stop” represents a stop codon, and WPRE represents a Woodchuck Hepatitis Virus (WHV) Posttranscriptional Regulatory Element.

FIGS. 7A and 7B provide plots showing the impact of AIP×5 administration on atrial fibrillation in a mouse model. LKB Flox/Flox mice were double injected with AAV-NPPA-Cre and AAV-AIP×5-mcherry (see FIG. 6). FIG. 7A provides plots showing results from an experiment where mice positive for the LKB1 flanked by LoxP sites (LKB1flox/flox) were injected with either AAV9-NPPA-RFP, AAV9-NPPA-Cre or double injections of AAV9-NPPA-Cre+AAV9-cTnT-AIP×5 at post-natal day 3 (P3). At two week intervals, ECG recordings for 3 minutes were performed under anesthesia. The time difference between two QRS complexes (RR interval) was plotted as a function of the following interval (RR+1). FIG. 7B provides a plot where the standard deviation of the RR intervals for a 1 minute recording was calculated for each animal at the time points shown.

FIG. 8 provides a schematic of the treatment and testing regimen for Example 5 disclosed herein.

FIG. 9 provides plots and graphs illustrating beat-to-beat variability in control mice as compared to LKB1 knockout mice. (A) shows that there was very little beat-to-beat variability in control mice (floxed LKB1 mice in the absence of Cre). (B) shows that there was a substantial increase in beat-to-beat variability in LKB1 knockout mice (floxed LKB1 mice in the presence of Cre) as compared to control mice.

FIG. 10 provides a plot showing that AIP×5 was associated with a reduction in beat-to-beat variability in LKB1 knockout mice.

FIG. 11 provides a plot showing that AIP×5 was associated with a reduction in beat-to-beat variability in Tbx5 knockout mice.

### DETAILED DESCRIPTION OF THE INVENTION

The invention of the disclosure generally features multimeric polypeptides that inhibit Ca²⁺-calmodulin dependent kinase II (CaMKII) (e.g., AIP multimeric polypeptide), polynucleotides encoding CaMKII inhibitory multimeric polypeptides, and methods of using the same for the treatment of a cardiac disease or disorder (e.g., catecholaminergic polymorphic ventricular tachycardia (CPVT) or atrial fibrillation (AF)).

As reported in detail below, the invention of the disclosure is based, at least in part, on the discovery that AIP multimeric polypeptides showed increased CaMKII inhibition and increased arrhythmia suppression relative to non-multimerized AIPs. Inhibition of CaMKII activation and subsequent downstream signaling significantly reduced the catecholamine-stimulated latent arrhythmia that is associated with mutations in the calcium ryanodine channel, RYR2. Accordingly, the invention of the disclosure provides compositions containing AIP multimers or polynucleotides encoding the same, and methods for use thereof in the treatment of cardiac diseases and disorders (e.g., CPVT or AF). In embodiments, the AIP multimeric polypeptides are delivered to a subject using an expression vector (e.g., AAV vector).

### Catecholaminergic Polymorphic Ventricular Tachycardia (CPVT)

Catecholaminergic polymorphic ventricular tachycardia (CPVT) is an inherited arrhythmia predominantly caused by autosomal dominant mutation of the gene encoding the cardiac ryanodine receptor (RYR2), the main intracellular calcium release channel of cardiomyocytes. Typically, CPVT patients are asymptomatic at rest but develop potentially lethal ventricular tachycardia during exercise or emotional distress. In wild type cardiomyocytes, when the cardiac action potential opens the voltage sensitive L-type Ca²⁺ channel located in the plasma membrane, the resulting local influx of Ca²⁺ triggers release of Ca²⁺ from the sarcoplasmic reticulum via RYR2. The resulting increase in cytoplasmic Ca²⁺ leads to sarcomere contraction. As the cell enters diastole, RYR2 closes and cytosolic Ca²⁺ is pumped back into the sarcoplasmic reticulum by the sarcoplasmic reticulum Ca²⁺-ATPase. In cells carrying mutations associated with CPVT, RYR2 releases more into the cytoplasm, resulting in elevated diastolic Ca²⁺ that drives exchange of sodium and calcium through the plasma membrane via the sodium calcium exchanger (NCX1), leading to after-depolarizations that may trigger additional action potentials. The molecular mechanism by which catecholamine stimulation unmasks the arrhythmic nature of CPVT mutations is not known. The mechanisms by which RYR2 mutation yields the clinical phenotype of ventricular tachycardia is also uncertain.

CPVT mutations increase diastolic Ca²⁺ release from the sarcoplasmic reticulum into the cytoplasm by RYR2. In individual cardiomyocytes, elevated diastolic Ca²⁺ induces reverse sodium-calcium exchange through NCX1 at the plasma membrane, resulting in after-depolarizations that potentially can trigger additional action potentials. The molecular mechanism by which catechol stimulation unmasks the arrhythmic nature of CPVT mutations is not known, although catechol-induced activation of Ca²⁺-calmodulin-dependent protein kinase II (CaMKII) has been implicated. The mechanisms by which RYR2 mutation yields the clinical phenotype of ventricular tachycardia is also uncertain, although one theory is that cardiomyocyte triggered activity produces ventricular tachycardia.

CPVT is associated with recurrent atrial or ventricular arrhythmias during exercise or emotional distress. The average age of onset is about 10 years of age, where 30% of patients present with cardiac arrest. From about 60% to about 70% of patients with CPVT have mutations in RYR2. CPVT is typically treated using medications, left cardiac sympathetic denervation (LCSD), or implantable cardioverter defibrillator (ICD) if episodes of atrial or ventricular arrhythmias are recurrent.

The advent of induced pluripotent stem cell (iPSC) technology and efficient methods to differentiate iPSCs to cardiomyocytes (iPSC-CMs) have created exciting opportunities to study inherited arrhythmias. iPSC-CMs have been generated from patients with CPVT as well as other inherited arrhythmias and have been shown to capture key features of these diseases, including abnormal action potential duration and drug responses.

### Atrial Fibrillation (AF)

Atrial fibrillation (AF or A-fib) is an abnormal heart rhythm (arrhythmia) characterized by rapid and irregular beating of the atrial chambers of the heart. It often begins as short periods of abnormal beating, which become longer or continuous over time. It may also start as other forms of arrhythmia such as atrial flutter that then transform into AF. Episodes can be asymptomatic. Symptomatic episodes may involve heart palpitations, fainting, lightheadedness, shortness of breath, or chest pain. Atrial fibrillation is associated with an increased risk of heart failure, dementia, and stroke. It is a type of supraventricular tachycardia.

Atrial fibrillation is the most common serious abnormal heart rhythm and, as of 2020, affects more than 33 million people worldwide. As of 2014, it affected about 2 to 3% of the population of Europe and North America. This was an increase from 0.4 to 1% of the population around 2005. In the developing world, about 0.6% of males and 0.4% of females are affected. The percentage of people with AF increases with age with 0.10% under 50 years old, 4% between 60 and 70 years old, and 14% over 80 years old being affected. A-fib and atrial flutter resulted in 193,300 deaths in 2015, up from 29,000 in 1990.

Atrial fibrillation can be diagnosed using an electrocardiogram, blood tests, a Holter monitor, an event recorder, an echocardiogram, a stress test, a chest X-ray, combinations thereof, etc. Typically, treatments for AF include medications (e.g., beta blockers, calcium channel blockers, digoxin, anti-arrhythmic medications, and/or blood thinners), cardioversion therapy (e.g., electrical cardioversion, or drug cardioversion), or surgery or catheter procedures (e.g., atrioventricular (AV) node ablation, or a maze procedure).

### Multimeric Polypeptides

CaMKII inhibitory multimeric polypeptides (e.g., multimeric polypeptides comprising AIP repeats) are those that significantly reduce CaMKII activity (e.g., CaMKII kinase activity, such as the phosphorylation of RYR2 by CaMKII).

In embodiments the multimeric polypeptides of the invention contain about or at least about 2, 3, 4, 5, 6, 7, 8, 9, 10, 11, 12, 13, 14, 15, 16, 17, 18, 19, or 20 repeats of a sequence containing, for example, about or at least about 5, 6, 7, 8, 9, 10, 11, 12, 13, 14, 15, 16, 17, 18, 19, or 20 amino acids of the sequence YKKALHRQEAVDAL (AIP) (SEQ ID NO: 15). In embodiments, the repeats are contiguous and adjacent to one another. In some instances, one or more of the repeats are separated by a linker, where the linker can be a stretch of amino acid residues that is about, at least about, and/or no more than about 1, 2, 3, 4, 5, 6, 7, 8, 9, or 10 amino acid residues in length.

In embodiments, a CaMKII inhibitory multimeric polypeptide (e.g., multimeric polypeptides comprising AIP repeats) reduces CaMKII activity by at least about 10, 25, 50, 75 or 100%. In some embodiments, a CaMKII inhibitory multimeric polypeptide (e.g., multimeric polypeptides comprising AIP repeats) renders CaMKII activity undetectable.

In embodiments, a CaMKII inhibitory multimeric polypeptide (e.g., multimeric polypeptides comprising AIP repeats) has an EC50 for inhibition of CaMKII that is lower than that of a non-multimeric CaMKII inhibitory polypeptide. In embodiments, the CaMKII inhibitory multimeric polypeptides (e.g., multimeric polypeptides comprising AIP repeats) have an EC5o for inhibition of CaMKII that is 1.5-fold, 2-fold, 3-fold, 4-fold, 5-fold, 10-fold, 20-fold, 30-fold, 40-fold, or 50-fold less than that of a reference peptide. In some instances the CaMKII inhibitory multimeric polypeptides (e.g., multimeric polypeptides comprising AIP repeats) have an EC50 for inhibition of CaMKII that is less than 80%, 70%, 60%, 50%, 40%, 30%, 20%, 10%, 1%, or 0.1% of that for non-multimeric CaMKII inhibitory peptide (e.g., AIP). In some cases, EC50 is measured using a bioluminescent and homogeneous ADP monitoring assay for kinases (ADP-GLO™), which is described in Zegzouti, et al. “ADP-Glo: A Bioluminescent and Homogeneous ADP Monitoring Assay for Kinases,” ASSAY and Drug Development Technologies, December 2009, 560-572, doi.org/10.1089/adt.2009.0222, the disclosure of which is incorporated herein by reference in its entirety for all purposes.

In embodiments, CaMKII inhibitory multimeric polypeptides (e.g., multimeric polypeptides comprising AIP) are modified in ways that enhance or do not inhibit their ability to modulate cardiac rhythm. In one embodiment, the invention provides methods for optimizing an amino acid sequence or nucleic acid sequence by producing an alteration. Such changes may include certain mutations, deletions, insertions, post-translational modifications, and tandem replication. In one preferred embodiment, the CaMKII inhibitory multimeric polypeptide (e.g., multimeric polypeptides comprising AIP repeats) amino acid sequence is modified to enhance protease resistance, particularly metalloprotease resistance. Accordingly, the invention further includes analogs of any naturally-occurring polypeptide of the invention. Analogs can differ from the naturally-occurring the polypeptide of the invention by amino acid sequence differences, by post-translational modifications, or by both. Analogs of the invention will generally exhibit at least 85%, more preferably 90%, and most preferably 95% or even 99% identity with all or part of a naturally-occurring amino, acid sequence of the invention. The length of sequence comparison is at least 10, 13, 15 amino acid residues. Again, in an exemplary approach to determining the degree of identity, a BLAST program may be used, with a probability score between e⁻³ and e⁻¹⁰⁰ indicating a closely related sequence. Modifications include in vivo and in vitro chemical derivatization of polypeptides, e.g., acetylation, carboxylation, phosphorylation, or glycosylation; such modifications may occur during polypeptide synthesis or processing or following treatment with isolated modifying enzymes. Analogs can also differ from the naturally-occurring polypeptides of the invention by alterations in primary sequence. These include genetic variants, both natural and induced (for example, resulting from random mutagenesis by irradiation or exposure to ethanemethylsulfate or by site-specific mutagenesis as described in Sambrook, Fritsch and Maniatis, Molecular Cloning: A Laboratory Manual (2d ed.), CSH Press, 1989, or Ausubel et al., supra). Also included are cyclized peptides, molecules, and analogs which contain residues other than L-amino acids, e.g., D-amino acids or non-naturally occurring or synthetic amino acids, e.g., beta or gamma amino acids.

In addition to full-length polypeptides, the invention also includes fragments of any one of the polypeptides of the invention. As used herein, the term “a fragment” means at least 5, 6, 7, 8, 9, 10, 11, 12, or 13 amino acids in length. Fragments of the invention can be generated by methods known to those skilled in the art or may result from normal protein processing (e.g., removal of amino acids from the nascent polypeptide that are not required for biological activity or removal of amino acids by alternative mRNA splicing or alternative protein processing events).

CaMKII inhibitory multimeric polypeptide (e.g., multimeric polypeptides comprising AIP) analogs may exceed the physiological activity of a native CaMKII inhibitory polypeptide (e.g., non-multimeric AIP). Methods of analog design are well known in the art, and synthesis of analogs can be carried out according to such methods by modifying the chemical structures such that the resultant analogs exhibit the activity of an unmodified CaMKII inhibitory multimeric polypeptide. These chemical modifications include, but are not limited to, substituting alternative R groups and varying the degree of saturation at specific carbon atoms of the CaMKII inhibitory multimeric polypeptide. Preferably, the CaMKII inhibitory multimeric polypeptide analogs are relatively resistant to in vivo degradation, resulting in a more prolonged therapeutic effect upon administration. Assays for measuring functional activity include, but are not limited to, those described in the Examples below.

### Polynucleotide Therapy

Polynucleotide therapy featuring a polynucleotide encoding a CaMKII inhibitory multimeric polypeptide (e.g., a multimer of AIP), analogs, variants, or fragments thereof is another therapeutic approach for treating a cardiac arrhythmia (e.g., CPVT or AF). Expression of such proteins in a cardiac cell is expected to modulate function of the cardiac cell, tissue, or organ, for example, by inhibiting phosphorylation of RYR2, inhibiting CaMKII activity, and/or otherwise regulating cardiac rhythm. Such nucleic acid molecules can be delivered to cells (e.g., cardiac cells) of a subject having a cardiac arrhythmia. The nucleic acid molecules are typically delivered to the cells of a subject in a form in which they can be taken up so that therapeutically effective levels of a CaMKII inhibitory multimeric polypeptide (e.g., AIP×3, AIP×5) or fragment thereof can be produced.

Transducing viral (e.g., retroviral, adenoviral, and adeno-associated viral (AAV)) vectors can be used for somatic cell gene therapy, especially because of their high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). For example, a polynucleotide encoding CaMKII inhibitory multimeric polypeptide (e.g., an AIP×5 multimer, variant, or a fragment thereof) can be cloned into a viral vector and expression can be driven from a promoter (e.g., a promoter specific for a target cell type of interest). In one embodiment, a viral vector (e.g., an AAV vector) is used to administer a polynucleotide encoding an AIP multimeric polypeptide (e.g., AIP×3 or AIP×5) to a cardiac tissue.

Transducing viral vectors have tissue tropisms that permit selective transduction of one cell type compared to another. For instance, while CaMKII inhibition in cardiomyocytes will be therapeutic for CPVT, AF, or other forms of heart disease, its inhibition in other tissues, such as the brain, may not be desirable. In some embodiments, vectors that target cardiomyocytes with high specificity compared to other cell types are used. This would allow specific cardiac targeting of the expression of the CaMKII inhibitor peptide molecule. This is because CaMKII inhibition in other non-cardiac cell can be deleterious. Among potential adeno-associated virus candidates are AAV9, AAV6, AAV2i8, AAVrh74, AAVrh10, MyoAAV, Anc80, and Anc82. Adeno-associated virus transduction efficiency is enhanced when the genome is “self-complementary.” In some embodiments, a self-complementary adeno-associated virus is used to increase the cardiac transduction by the gene therapy vector.

Other viral vectors that can be used include, for example, a vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; Le Gal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346).

Non-viral approaches can also be employed for the introduction of therapeutic to a cardiac cell of a patient requiring inhibition of CaMKII. For example, a nucleic acid molecule can be introduced into a cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Preferably the nucleic acids are administered in combination with a liposome and protamine.

Gene transfer can also be achieved using non-viral means involving transfection in vitro. Such methods include the use of calcium phosphate, DEAE dextran, electroporation, and protoplast fusion. Liposomes or lipid nanoparticles can also be potentially beneficial for delivery of DNA into a cell. Transplantation of normal genes into the affected tissues of a patient can also be accomplished by transferring a normal nucleic acid into a cultivatable cell type ex vivo (e.g., an autologous or heterologous primary cell or progeny thereof), after which the cell (or its descendants) are injected into a targeted tissue.

The dosage of the administered vectors disclosed herein depends on a number of factors, including the size and health of the individual patient. For any particular subject, the specific dosage regimes should be adjusted over time according to the individual need and the professional judgment of the person administering or supervising the administration of the compositions. In some embodiments, the dosage is between about 1×10¹⁰ vector units/kg to about 1×10¹⁴ vector units/kg. In some embodiments, the dosage is about 1×10¹⁰ vector units/kg, about 5×10¹⁰ vector units/kg, about 1×10¹¹ vector units/kg, about 5×10¹¹ vector units/kg, about 1×10¹² vector units/kg, about 5×10¹² vector units/kg, about 1×10¹³ vector units/kg, about 5×10¹³ vector units/kg, or about 1×10¹⁴ vector units/kg. In an exemplary embodiment, the vector is a viral vector, and the dosage is between about 1×10¹⁰ viral genomes/kg to about 1×10¹⁴ viral genomes/kg. In some embodiments, dosage is based on transfection, transformation, or transduction efficiency. In some embodiments, the dosage is effective to transfect, transform, or transduce at least about 20% of cardiomyocytes in a subject, at least about 25% of cardiomyocytes in a subject, at least about 30% of cardiomyocytes in a subject, at least about 35% of cardiomyocytes in a subject, or at least about 40% of cardiomyocytes in a subject. In some embodiments, the dosage is effective to transfect, transform, or transduce between about 20% of myocytes in a subject to about 40% of myocytes in a subject.

Transcription of mRNA from a polynucleotide of the invention (e.g., a polynucleotide encoding a CaMKII inhibitory multimeric polypeptide (e.g., a multimer of AIP)) can be directed from any suitable promoter (e.g., the human cytomegalovirus (CMV), simian virus 40 (SV40), the CMV-chicken b-actin hybrid promoter (“CAG”), or metallothionein promoters, and regulated by any appropriate mammalian regulatory element. For treatment of CPVT or AF, it is desirable to selectively express a CaMKII inhibitory multimeric polypeptide (e.g., a multimer of AIP) in cardiomyocytes and to minimize expression in other cell types. In some embodiments, cardiomyocyte-selective promoters are used for the expression of the CaMKII inhibitory multimeric polypeptide (e.g., a multimer of AIP). The promoters or enhancers used can include, without limitation, those that are characterized as tissue- or cell-specific enhancers. For example, the cardiac troponin T promoter, the α-myosin heavy chain (α-MHC) promoter, the myosin light chain-2v (MLC-2v) promoter or the cardiac NCX1 promoter can be used to direct expression in cardiomyocytes. Alternatively, if a genomic clone is used as a therapeutic construct, regulation can be mediated by the cognate regulatory sequences or, if desired, by regulatory sequences derived from a heterologous source, including any of the promoters or regulatory elements described above.

Another therapeutic approach included in the invention of the disclosure involves administration of a recombinant CaMKII inhibitory multimeric polypeptide (e.g., a multimer of AIP), such as a recombinant AIP multimeric polypeptide (e.g., AIP×3 or AIP×5), variant, or fragment thereof, either directly to a site of a potential or actual disease-affected tissue or systemically (for example, by any conventional recombinant protein administration technique). The dosage of the administered peptide depends on a number of factors, including the size and health of the individual patient. For any particular subject, the specific dosage regimes should be adjusted over time according to the individual need and the professional judgment of the person administering or supervising the administration of the compositions.

Stabilized mRNA

Stabilized mRNA (e.g., mRNA stabilized by increasing or improving half-life, increasing or improving potency, increasing or improving resistance to endonuclease activity, decreasing or reducing susceptibility to endonuclease activity) encoding CaMKII inhibitory multimeric polypeptides are useful in some embodiments for preventing or ameliorating CPVT, AF, or another cardiac arrhythmia. Such stabilized mRNA can be delivered to cells (e.g., cardiac cells) of a subject having a cardiac arrhythmia. (e.g., CPVT or AF).

Expression of the CaMKII inhibitory multimeric polypeptides encoded by the stabilized mRNA described herein, in a cardiac cell, is expected to modulate function of the cardiac cell, tissue, or organ, for example, by inhibiting phosphorylation of RYR2, inhibiting CaMKII activity, and/or otherwise regulating cardiac rhythm. In some embodiments, the stabilized mRNA encodes a multimeric AIP polypeptide, as disclosed herein.

### Therapeutic Methods

The CaMKII inhibitory multimeric polypeptides (e.g., a multimer of AIP) and polynucleotides or vectors (e.g., an AAV vector) encoding the same are useful for preventing or ameliorating CPVT, AF, or another cardiac arrhythmia. Diseases and disorders characterized by cardiac arrhythmia may be treated using the methods and compositions of the invention (e.g., atrial fibrillation (AF), catecholaminergic polymorphic ventricular tachycardia (CPVT), ischemic heart disease, cardiac arrhythmia, heart failure (e.g., heart failure associated with aortic binding), hypertensive heart disease and pulmonary hypertensive heart disease, myocardial infarction, valvular disease, congenital heart disease, myocardial hypertrophy, ventricular arrhythmia, or Timothy syndrome).

In one therapeutic approach, an agent identified as described herein is administered to the site of a potential or actual disease-affected tissue or is administered systemically. The dosage of the administered agent depends on a number of factors, including the size and health of the individual patient. For any particular subject, the specific dosage regimes should be adjusted over time according to the individual need and the professional judgement of the person administering or supervising the administration of the compositions.

Delivery or transfer of the polypeptides, polynucleotides, or vectors, of the present invention may also be accomplished through the use of muscle targeting agents (e.g., agents which specifically target cardiac muscle). Exemplary muscle targeting agents, preferably cardiac muscle targeting agents, may be found in, for example, U.S. Pat. No. 11,168,141, filed Aug. 2, 2019, the contents of which are hereby incorporated by reference in their entirety. In some embodiments, muscle targeting agents (e.g., agents, such as antibodies, which target cardiac markers, such as Caveolin-3) may be conjugated to the polypeptides, polynucleotides, or vectors disclosed herein in order to target such polypeptides, polynucleotides, or vectors to targets in cardiac muscle.

### Pharmaceutical Compositions

The present disclosure provides pharmaceutical compositions containing a CaMKII inhibitory multimeric polypeptide (e.g., a multimer of AIP), or polynucleotides, and/or vectors encoding the same. The administration of a pharmaceutical composition of the present disclosure for the treatment of cardiac arrhythmia may be by any suitable means that results in a concentration of a CaMKII inhibitory multimeric polypeptide (e.g., a multimer of AIP) that is effective in ameliorating, reducing, or stabilizing a cardiac arrhythmia. Diseases and disorders characterized by cardiac arrhythmia (e.g., atrial fibrillation (AF), catecholaminergic polymorphic ventricular tachycardia (CPVT), ischemic heart disease, cardiac arrhythmia, heart failure (e.g., heart failure associated with aortic binding), hypertensive heart disease and pulmonary hypertensive heart disease, myocardial infarction, valvular disease, congenital heart disease, myocardial hypertrophy, ventricular arrhythmia, or Timothy syndrome) may be treated using the pharmaceutical compositions of the invention. The composition may be contained in any appropriate amount in any suitable carrier substance. The composition may be provided in a dosage form that is suitable for parenteral (e.g., subcutaneously, intravenously, intramuscularly, or intraperitoneally) administration route. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

For therapeutic uses, the polypeptides, polynucleotides, and/or vectors disclosed herein may be administered systemically, for example, formulated in a pharmaceutically-acceptable buffer, such as physiological saline. Preferable routes of administration include, for example, subcutaneous, intravenous, interperitoneally, intramuscular, or intradermal injections that provide continuous, sustained levels of the drug in the patient. For AAV gene therapy, administration may be local or systemic, e.g., intravenous or intracoronary. Treatment of human patients or other animals will be carried out using a therapeutically effective amount of a therapeutic identified herein in a physiologically-acceptable carrier. Suitable carriers and their formulation are described, for example, in Remington's Pharmaceutical Sciences by E. W. Martin. The amount of the therapeutic agent to be administered varies depending upon the manner of administration, the age and body weight of the patient, and with the clinical symptoms of the cardiac arrhythmia. Generally, amounts will be in the range of those used for other agents used in the treatment of other diseases requiring regulation of cardiac function, although in certain instances lower amounts will be needed because of the increased specificity of the compound. A composition comprising a CaMKII inhibitory multimeric polypeptide (e.g., a multimer of AIP) is administered at a dosage having CaMKII inhibitory activity or cardiac rhythm regulatory activity as determined by a method known to one skilled in the art, or using any that assay that measures the expression or the biological activity of a CaMKII polypeptide.

Pharmaceutical compositions according to the invention may be formulated to release the active compound substantially immediately upon administration or at any predetermined time or time period after administration. The latter types of compositions are generally known as controlled release formulations, which include (i) formulations that create a substantially constant concentration of the drug within the body over an extended period of time; (ii) formulations that after a predetermined lag time create a substantially constant concentration of the drug within the body over an extended period of time; (iii) formulations that sustain action during a predetermined time period by maintaining a relatively, constant, effective level in the body with concomitant minimization of undesirable side effects associated with fluctuations in the plasma level of the active substance (sawtooth kinetic pattern); (iv) formulations that localize action by, e.g., spatial placement of a controlled release composition adjacent to or in contact with the thymus; (v) formulations that allow for convenient dosing, such that doses are administered, for example, once every one or two weeks; and (vi) formulations that target a cardiac arrhythmia by using carriers or chemical derivatives to deliver the therapeutic agent to a particular cell type (e.g., cardiac cell). For some applications, controlled release formulations obviate the need for frequent dosing during the day to sustain the plasma level at a therapeutic level.

Any of a number of strategies can be pursued in order to obtain controlled release in which the rate of release outweighs the rate of metabolism of the compound in question. In one example, controlled release is obtained by appropriate selection of various formulation parameters and ingredients, including, e.g., various types of controlled release compositions and coatings. Thus, the therapeutic is formulated with appropriate excipients into a pharmaceutical composition that, upon administration, releases the therapeutic in a controlled manner. Examples include single or multiple unit tablet or capsule compositions, oil solutions, suspensions, emulsions, microcapsules, microspheres, molecular complexes, nanoparticles, patches, and liposomes.

### Parenteral Compositions

The pharmaceutical composition comprising a CaMKII inhibitory multimeric polypeptide (e.g., a multimer of AIP) of the present disclosure or a polynucleotide encoding the same, or a vector (e.g., an AAV vector containing the polynucleotide encoding the multimeric polypeptide) may be administered parenterally by injection, infusion or implantation (subcutaneous, intravenous, intramuscular, intraperitoneal, intracoronary or the like) in dosage forms, formulations, or via suitable delivery devices or implants containing conventional, non-toxic pharmaceutically acceptable carriers and adjuvants. In embodiments, the pharmaceutical composition contains a viral vector (e.g., an AAV vector) containing a polynucleotide encoding a CaMKII inhibitory multimeric polypeptide. The formulation and preparation of such compositions are well known to those skilled in the art of pharmaceutical formulation. Formulations can be found in Remington: The Science and Practice of Pharmacy, supra.

Compositions for parenteral use may be provided in unit dosage forms (e.g., in single-dose ampoules), or in vials containing several doses and in which a suitable preservative may be added (see below). The composition may be in the form of a solution, a suspension, an emulsion, an infusion device, or a delivery device for implantation, or it may be presented as a dry powder to be reconstituted with water or another suitable vehicle before use. Apart from the active agent that reduces or ameliorates a cardiac arrhythmia, the composition may include suitable parenterally acceptable carriers and/or excipients. The active therapeutic agent(s) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing, agents.

As indicated above, the pharmaceutical compositions according to the invention may be in the form suitable for sterile injection. To prepare such a composition, the suitable therapeutic(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. The aqueous formulation may also contain one or more preservatives (e.g., methyl, ethyl or n-propyl p-hydroxybenzoate). In cases where one of the compounds is only sparingly or slightly soluble in water, a dissolution enhancing or solubilizing agent can be added, or the solvent may include 10-60% w/w of propylene glycol or the like.

### Controlled Release Parenteral Compositions

Controlled release parenteral compositions may be in form of aqueous suspensions, microspheres, microcapsules, magnetic microspheres, oil solutions, oil suspensions, or emulsions. Alternatively, the active drug may be incorporated in biocompatible carriers, liposomes, nanoparticles, implants, or infusion devices.

Materials for use in the preparation of microspheres and/or microcapsules are, e.g., biodegradable/bioerodible polymers such as polygalactin, poly-(isobutyl cyanoacrylate), poly(2-hydroxyethyl-L-glutam-nine) and, poly(lactic acid). Biocompatible carriers that may be used when formulating a controlled release parenteral formulation are carbohydrates (e.g., dextrans), proteins (e.g., albumin), lipoproteins, or antibodies. Materials for use in implants can be non-biodegradable (e.g., polydimethyl siloxane) or biodegradable (e.g., poly(caprolactone), poly(lactic acid), poly(glycolic acid) or poly(ortho esters) or combinations thereof).

### Solid Dosage Forms For Oral Use

Formulations for oral use include tablets containing the active ingredient(s) in a mixture with non-toxic pharmaceutically acceptable excipients. Such formulations are known to the skilled artisan. Excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

The tablets may be uncoated or they may be coated by known techniques, optionally to delay disintegration and absorption in the gastrointestinal tract and thereby providing a sustained action over a longer period. The coating may be adapted to release the active drug in a predetermined pattern (e.g., in order to achieve a controlled release formulation) or it may be adapted not to release the active drug until after passage of the stomach (enteric coating). The coating may be a sugar coating, a film coating (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or an enteric coating (e.g., based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose). Furthermore, a time delay material, such as, e.g., glyceryl monostearate or glyceryl distearate may be employed.

The solid tablet compositions may include a coating adapted to protect the composition from unwanted chemical changes, (e.g., chemical degradation prior to the release of the active a cardiac active therapeutic substance). The coating may be applied on the solid dosage form in a similar manner as that described in Encyclopedia of Pharmaceutical Technology, supra.

In one embodiment, two or more cardiac therapeutics may be mixed together in the tablet, or may be partitioned. In one example, the first active cardiac therapeutic is contained on the inside of the tablet, and the second active therapeutic is on the outside, such that a substantial portion of the second therapeutic is released prior to the release of the first active therapeutic.

Formulations for oral use may also be presented as chewable tablets, or as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders and granulates may be prepared using the ingredients mentioned above under tablets and capsules in a conventional manner using, e.g., a mixer, a fluid bed apparatus or a spray drying equipment.

Controlled Release Oral Dosage Forms Controlled release compositions for oral use may, e.g., be constructed to release the AIP multimer or CaM-KNtide multimers, vectors, and/or polynucleotides of the present disclosure by controlling the dissolution and/or the diffusion thereof. Dissolution or diffusion controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated metylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

A controlled release composition containing one or more therapeutic compounds may also be in the form of a buoyant tablet or capsule (i.e., a tablet or capsule that, upon oral administration, floats on top of the gastric content for a certain period of time). A buoyant tablet formulation of the compound(s) can be prepared by granulating a mixture of the compound(s) with excipients and 20-75% w/w of hydrocolloids, such as hydroxyethylcellulose, hydroxypropylcellulose, or hydroxypropylmethylcellulose. The obtained granules can then be compressed into tablets. On contact with the gastric juice, the tablet forms a substantially water-impermeable gel barrier around its surface. This gel barrier takes part in maintaining a density of less than one, thereby allowing the tablet to remain buoyant in the gastric juice.

### Combination Therapies

Optionally, a CaMKII inhibitory multimeric polypeptide (e.g., a multimer of AIP) described herein may be administered in combination with any other standard therapy useful for regulating cardiac function; such methods are known to the skilled artisan and described in Remington's Pharmaceutical Sciences by E. W. Martin.

### Kits

Also provided are kits for preventing or treating a cardiac arrhythmia, cardiac condition, or pathology (e.g., atrial fibrillation (AF), catecholaminergic polymorphic ventricular tachycardia (CPVT), ischemic heart disease, cardiac arrhythmia, heart failure (e.g., heart failure associated with aortic binding), hypertensive heart disease and pulmonary hypertensive heart disease, myocardial infarction, valvular disease, congenital heart disease, myocardial hypertrophy, ventricular arrhythmia, or Timothy syndrome) in a subject in need thereof. In one embodiment, the kit provides a therapeutic or prophylactic composition containing an effective amount of a CaMKII inhibitory multimeric polypeptide (e.g., a multimer of AIP), a polynucleotide encoding such polypeptide, and/or a vector comprising such polynucleotide, where the kit is for use in administering the multimeric polypeptide, polynucleotide, or vector to a subject.

In another embodiment, the kit provides a therapeutic or prophylactic composition containing an effective amount of a CaMKII inhibitory multimeric polypeptide (e.g., a multimer of AIP), polynucleotide, and/or vector encoding the same.

In some embodiments, the kit comprises a sterile container which contains the therapeutic or prophylactic composition; such containers can be boxes, ampoules, bottles, vials, tubes, bags, pouches, blister-packs, or other suitable container forms known in the art. The containers can be made of plastic, glass, laminated paper, metal foil, or other materials suitable for holding medicaments.

In embodiments, the kit contains instructions for administering the composition to a subject having or at risk of developing a disease (e.g., CPVT or AF). The instructions will generally include information about the use of the composition for the treatment of the disease. In other embodiments, the instructions include at least one of the following: description of the therapeutic agent; dosage schedule and administration for treatment or prevention of a cardiac disease (e.g., CPVT or AF) or symptoms thereof; precautions; warnings; indications; counter-indications; overdosage information; adverse reactions; animal pharmacology; clinical studies; and/or references. The instructions may be printed directly on the container (when present), or as a label applied to the container, as information stored on a remotely-accessible server, or as a separate sheet, pamphlet, card, or folder supplied in or with the container.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1: Multimerization Improved Potency of Inhibition of CaMKII Inhibition by Autocamtide-2-Related Inhibitory Peptide (AIP) In Vivo, but not of an Alternative Potent Inhibitor of CaMKII

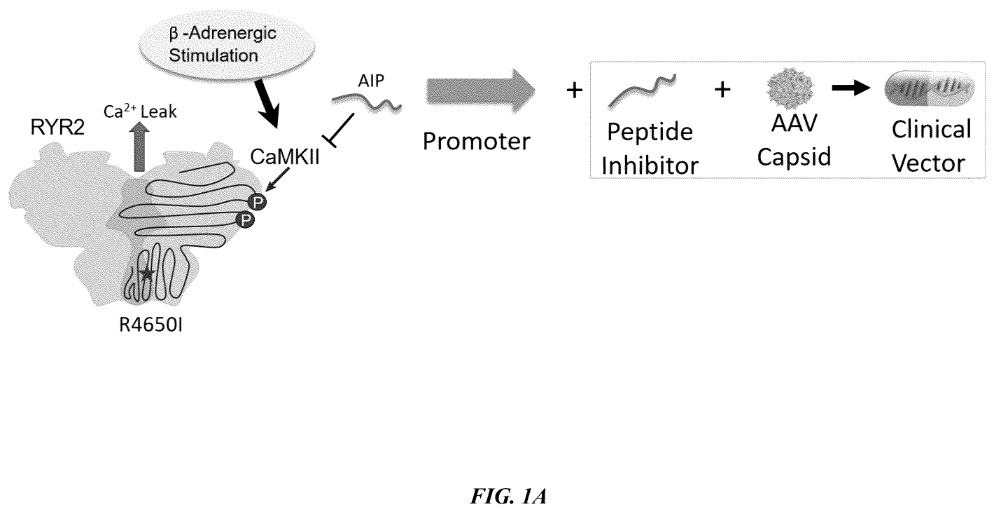

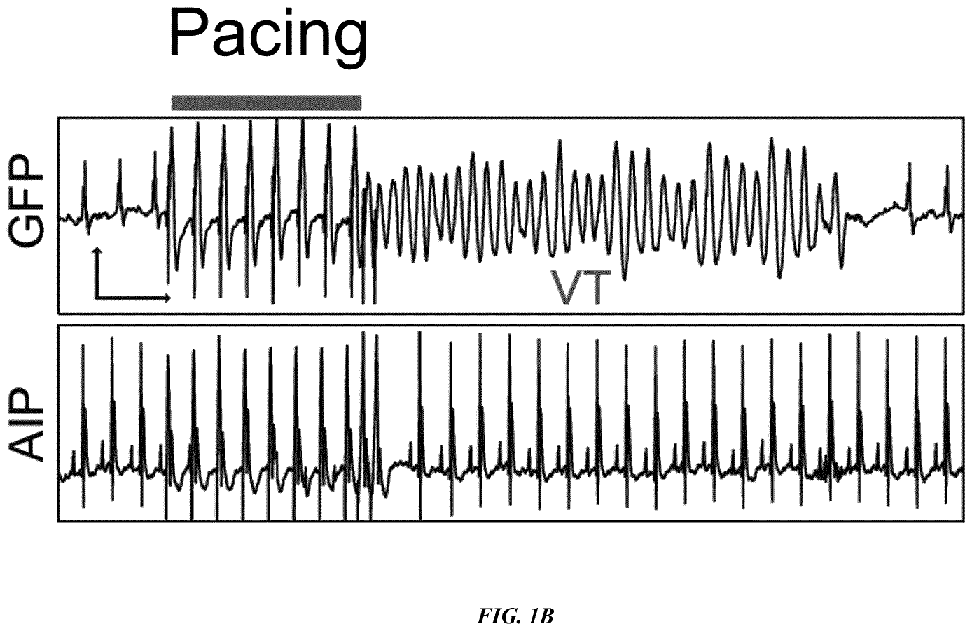

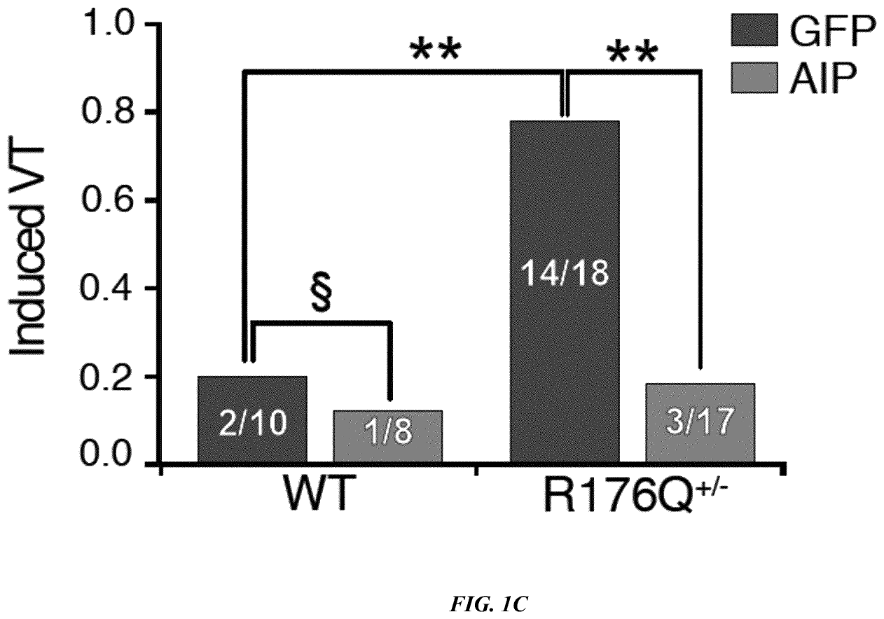

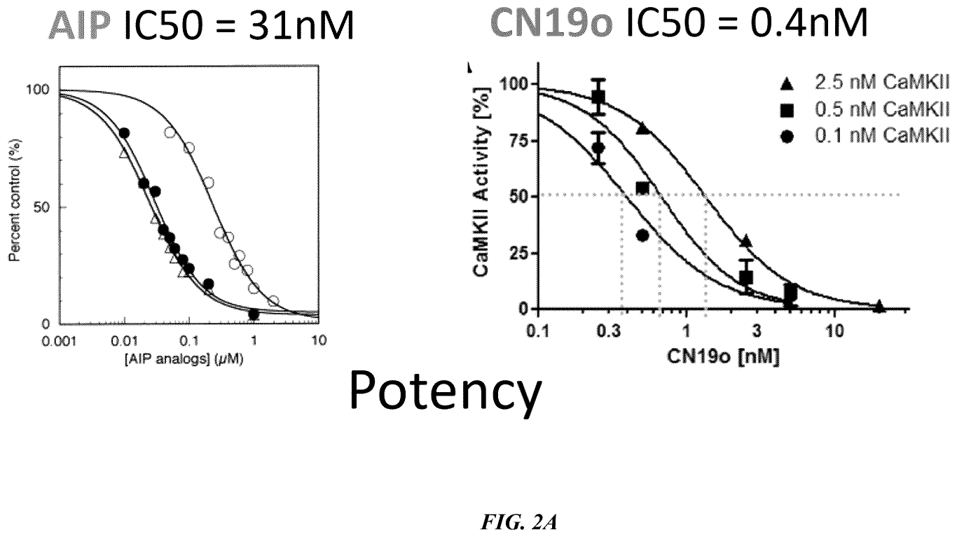

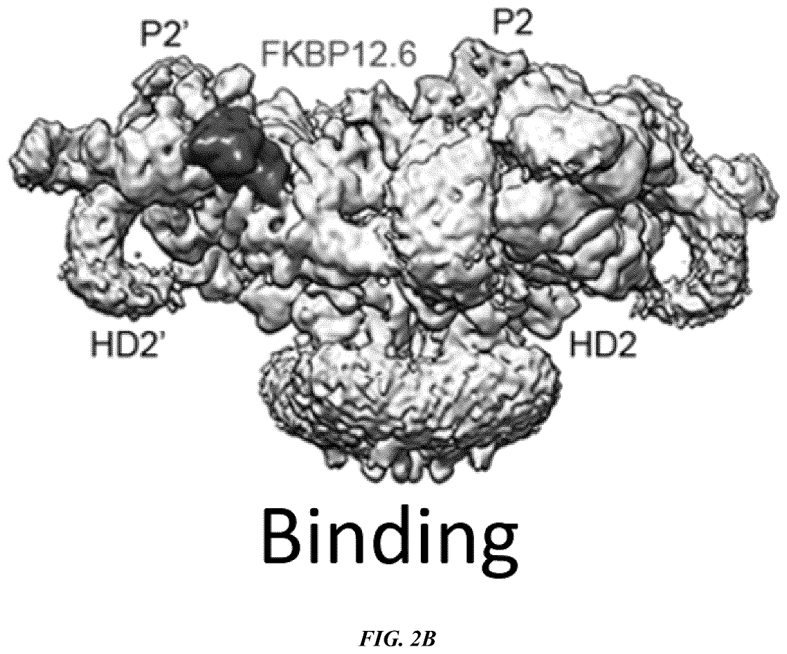

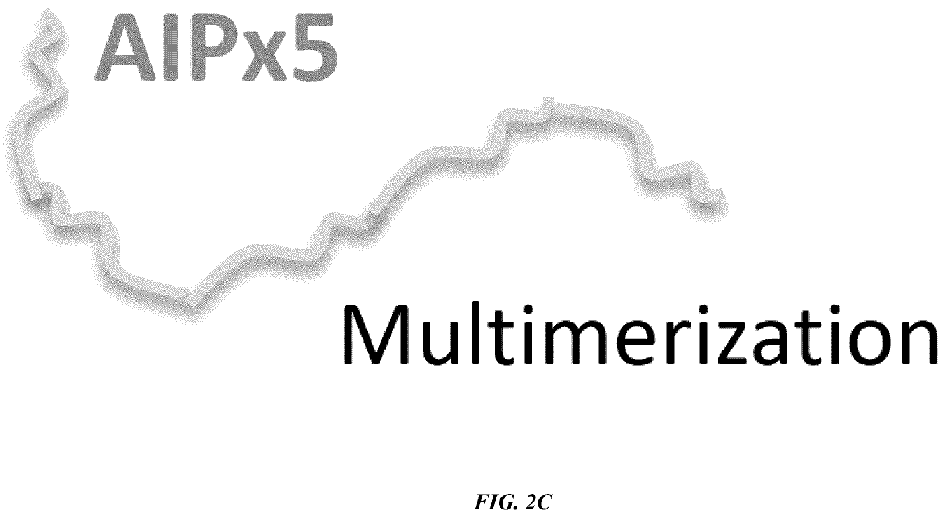

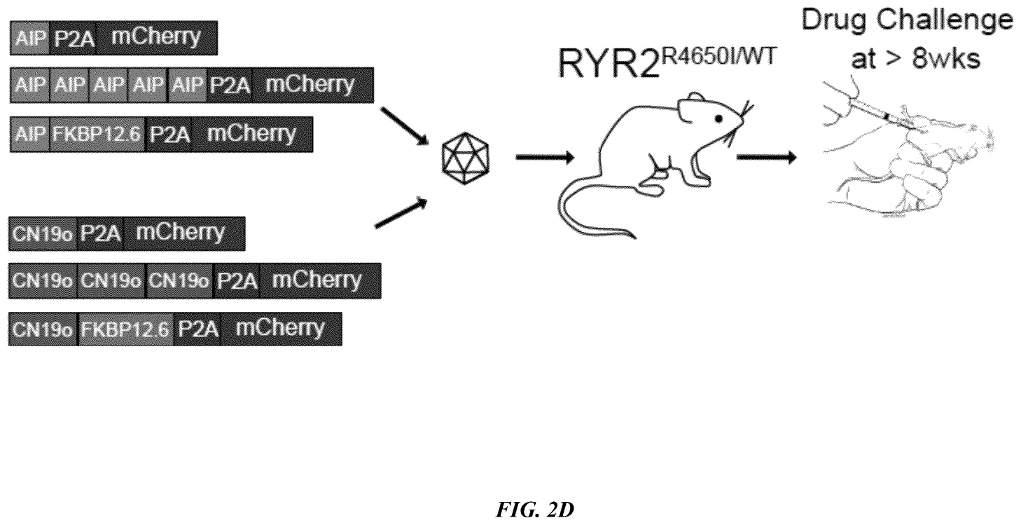

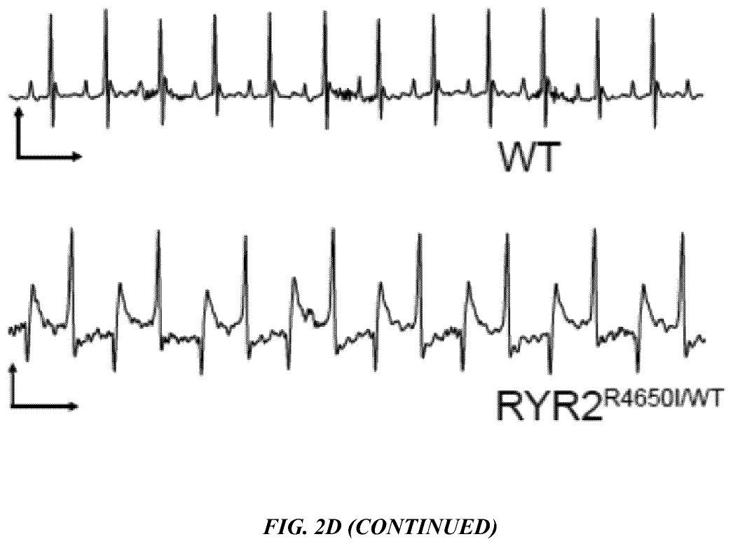

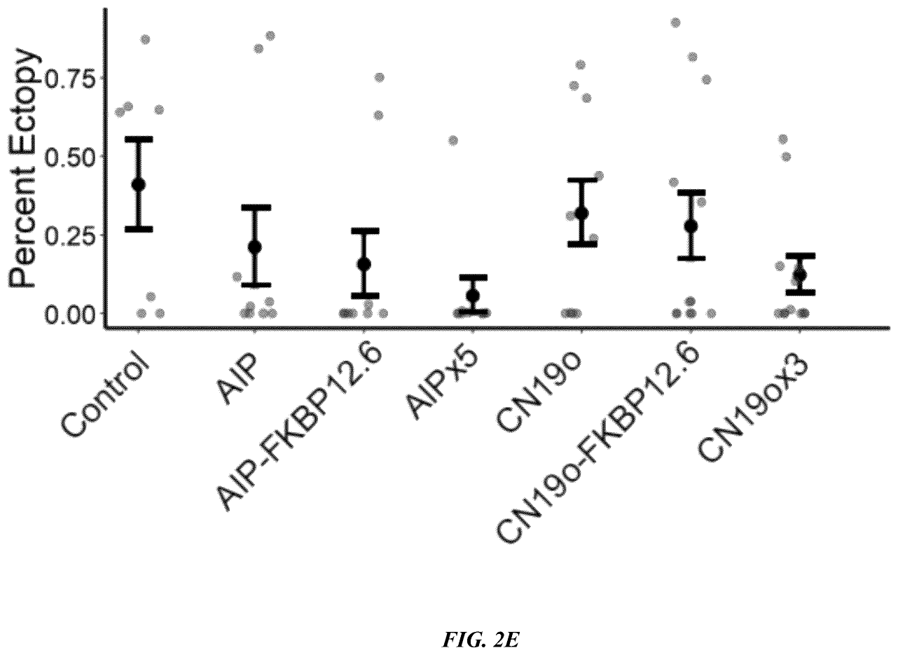

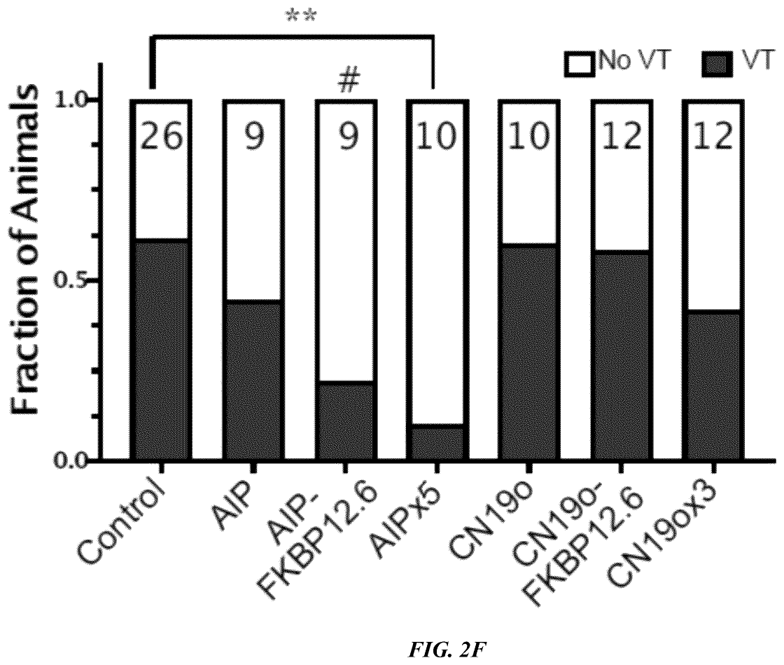

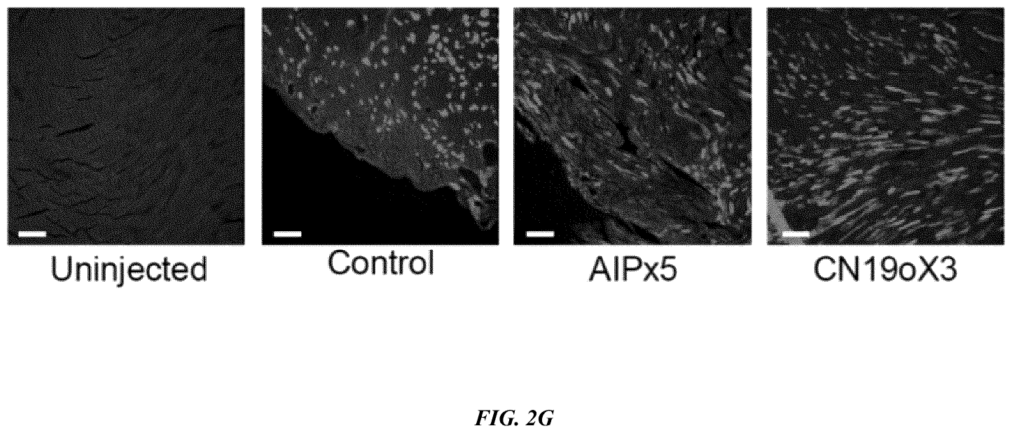

Experiments were undertaken to identify improved agents for use in the treatment of catecholaminergic polymorphic ventricular tachycardia (CPVT). The peptide inhibitor autocamtide-2-related inhibitory peptide (AIP) is effective in the treatment of CPVT (see FIGS. 1A-1C). To identify improved agents for the treatment of CPVT, a multimeric peptide comprising five consecutive repeats of AIP (i.e., AIP×5) was designed and the efficacy of the peptide in inhibiting the activity of the kinase CaMKII was evaluated (see FIGS. 2A-2G, 3, 4, and 5A-5E).

The development of improved agents for the treatment of CPVT, the CPVT treatment efficacy of polypeptides A) containing multiple repeats of AIP (FIG. 2C) or the potent CaMKII inhibitor CN19o or B) containing AIP or CN19o fused to FKBP12.6 (FIG. 2B), which binds RYR2, which is a target of CaMKII (FIG. 1A). The domain architectures of the polypeptides evaluated are presented in FIG. 2D. CN19o is known to be a more potent inhibitor of CaMKII than AIP (FIG. 2A). The efficacy of the polypeptides in treatment of catecholaminergic polymorphic ventricular tachycardia (CPVT) was evaluated in the RYR2^{R4650I/WT} mouse model of CPVT as shown in FIG. 2D. Of all peptides evaluated, the AIP×5 peptide was found to be the most effective in treating CPVT, as determined by observed reductions in ectopy (FIGS. 2E and 2F). AIP-FKB12.6 was also effective in treating symptoms of CPVT by reducing ectopy (FIGS. 2E and 2F). The polypeptides were administered to the mice using an AAV vector and delivery of polynucleotides expressing the polypeptides to cardiac tissue was confirmed using fluorescent imaging (FIG. 2G).

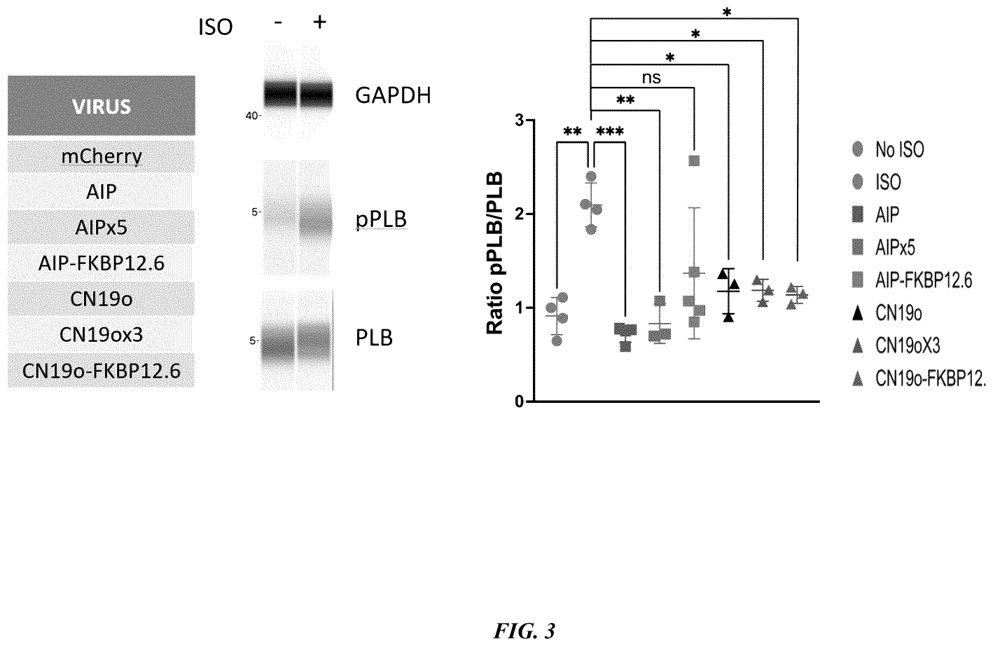

To confirm suppression of CaMKII activation in vivo, phosphorylation of phospholamban (PLB) at threonine-17, a known target of CaMKII, was evaluated in heart tissue from mice and whole heart lysates exposed to isoproterenol (ISO) (FIG. 3). Exposure of cardiac cells to ISO induced phosphorylation of PLB by CaMKII (FIG. 3). It was determined that contacting cells with AIP or AIP×5 resulted in a statistically significant decrease in PLB phosphorylation in cells exposed to ISO.

### Example 2: Multimerization Improved Potency of Inhibition of CaMKII Inhibition by Autocamtide-2-Related Inhibitory Peptide (AIP) In Vitro, but not of an Alternative Potent Inhibitor of CaMKII

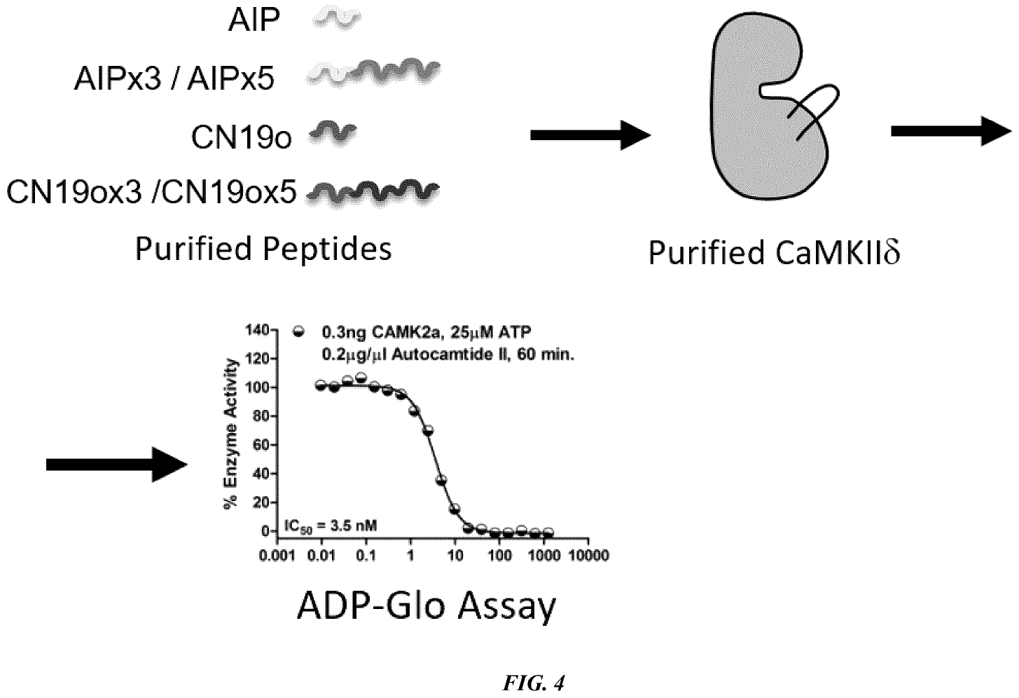

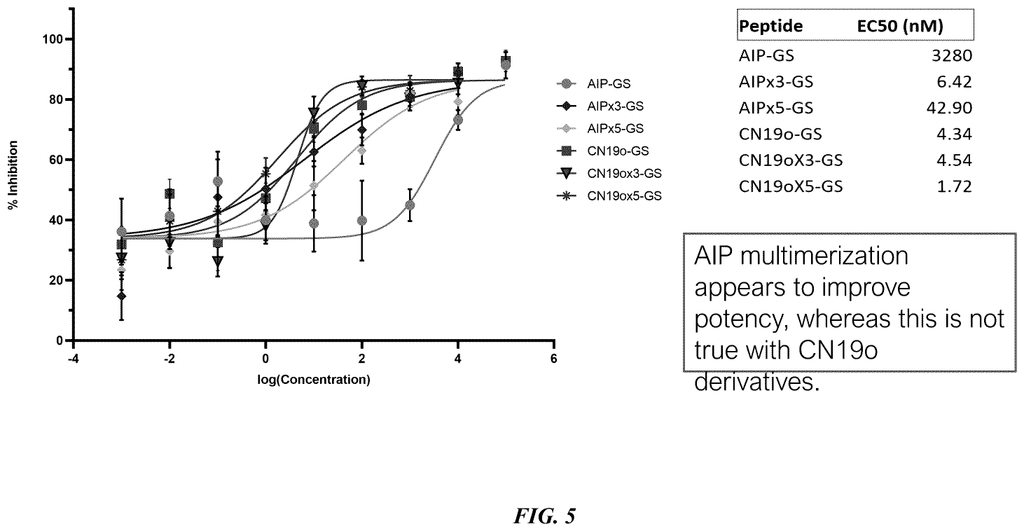

Experiments were undertaken to evaluate in vitro the inhibition of CaMKIIδ by the polypeptides evaluated in Example 1, the amino acid sequences of which are provided in Table 1 below. Inhibition of CaMKIIδ activity by the polypeptides was evaluated using a bioluminescent and homogeneous ADP monitoring assay for kinases (ADP-GLO™), which is described in Zegzouti, et al. “ADP-Glo: A Bioluminescent and Homogeneous ADP Monitoring Assay for Kinases,” ASSAY and Drug Development Technologies, December 2009, 560-572, doi.org/10.1089/adt.2009.0222, the disclosure of which is incorporated herein by reference in its entirety for all purposes (FIG. 4). Multimerization (i.e., repeats of the AIP peptide or the CN19oX5 peptide) was found to increase potency (i.e., lower EC50) for AIP but to actually decrease potency (i.e., raise EC50) for CN19oX5 (FIGS. 5B-5E). The EC50 for AIP was 6149 nM, which was reduced to 543 nM for an AIP×3 multimer and reduced even further to 114 nM for the AIP×5 multimer. However, the EC50 value of CN19o of 1.6 nM was increased to 2.1 nM in the CN19oX3 multimer. Therefore, multimerization increased the potency of AIP, but actually decreased the potency of CN19oX3.

TABLE 1
Peptide sequences.

| Name | Sequence | Length |
|---|---|---|
| CN190 | KRAPKLGQIGROKAVDIED | 19 |
| | (SEQ ID NO: 2) | |
| AIP | YKKALHRQEAVDAL | 14 |
| | (SEQ ID NO: 15) | |
| CN19oX3 | KRAPKLGQIGROKAVDIEDKRAPKLGQIGRQKAVD | 57 |
| | IEDKRAPKLGQIGROKAVDIED | |
| | (SEQ ID NO: 9) | |
| AIPx3 | YKKALHRQEAVDALYKKALHRQEAVDALYKKALHR | 42 |
| | QEAVDAL | |
| | (SEQ ID NO: 3) | |
| CN19oX5 | KRAPKLGQIGRQKAVDIEDKRAPKLGQIGRQKAVD | 95 |
| | IEDKRAPKLGQIGRQKAVDIEDKRAPKLGQIGRQK | |
| | AVDIEDKRAPKLGQIGRQKAVDIED | |
| | (SEQ ID NO: 10) | |
| AIPx5 | YKKALHRQEAVDALYKKALHRQEAVDALYKKALHR | 70 |
| | QEAVDALYKKALHRQEAVDALYKKALHRQEAVDAL | |
| | (SEQ ID NO: 4) | |

### Example 3: Administration of AIP×5 to Animal Models

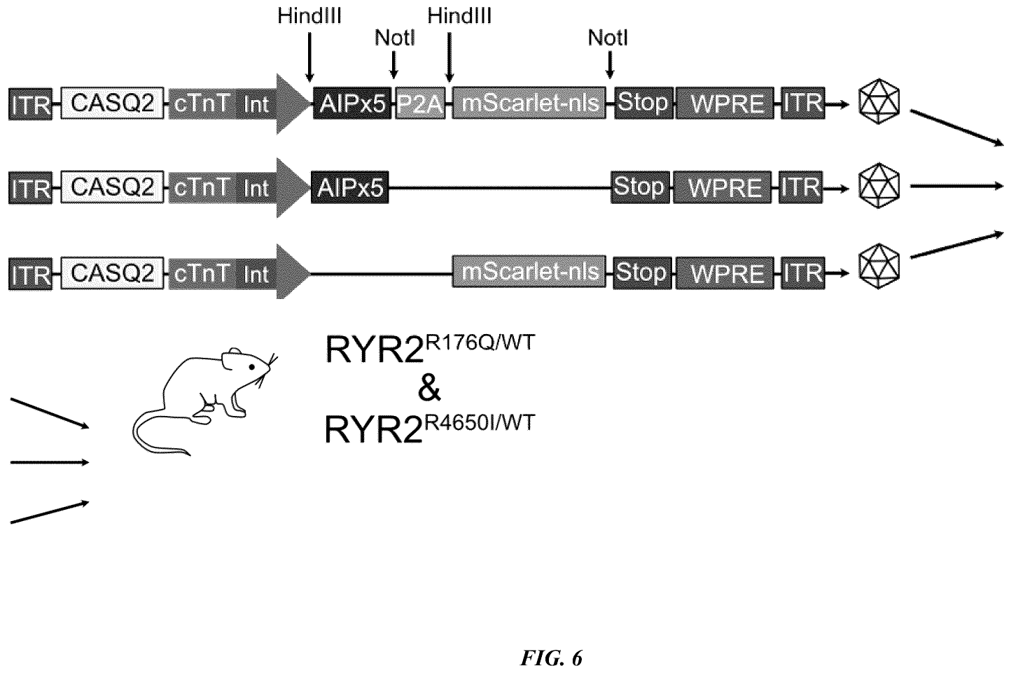

AAV vectors expressing AIP×5 or a negative control AIV vector expressing only mScarlet (see FIG. 6) are prepared and administered to RYR2^{R176Q/QT} and RYR2^{R4560I/WT} mice, which are animal models of catecholaminergic polymorphic ventricular tachycardia (CPVT). The efficacy of the vectors in reducing symptoms of CPVT is evaluated in the animal models. Administration of the AIP×5 vectors results in expression of AIP×5 in cardiac tissue of the mice and in a reduction in symptoms of CPVT (e.g., a reduction in ectopy and/or ventricular tachycardia).

### Example 4: AAV9-AIP×5 Suppressed Arrhythmia in an Animal Model of Atrial Fibrillation (AF)

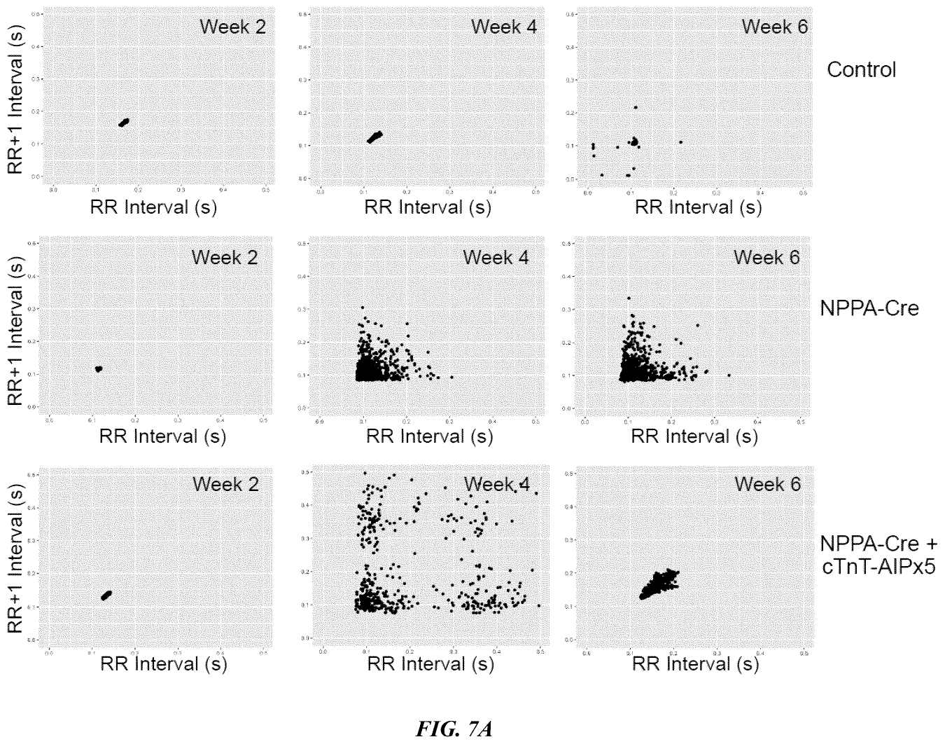

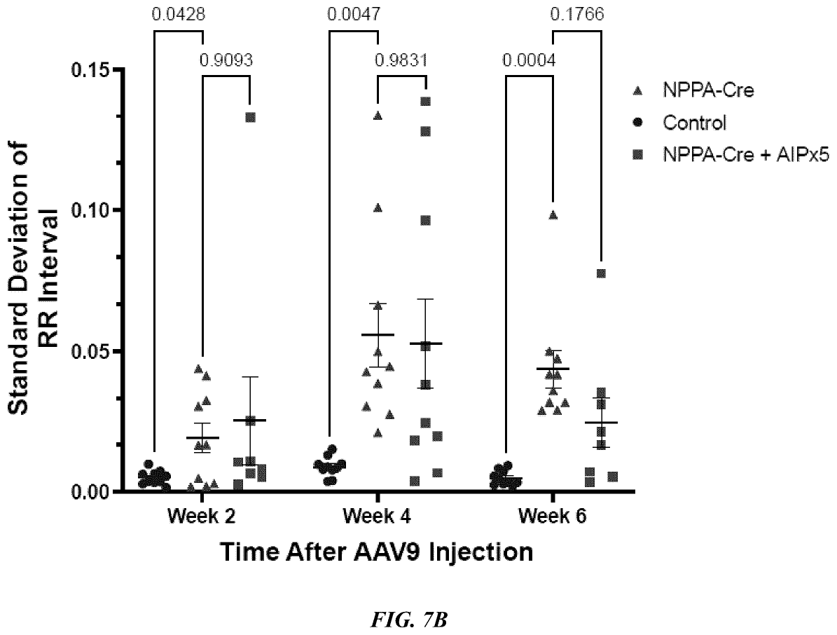

Experiments were undertaken to evaluate the efficacy of AIP×5 administration in treatment of atrial fibrillation. LKB Flox/Flox mice were double injected with NPPA-Cre and AIP×5-mcherry using an AAV vector. The mice were a model for atrial fibrillation. In the mouse model, NPPA-Cre inactivated LKB in atria, creating a model of atrial fibrillation (AF). It was evaluated whether co-injection with AAV-AIP×5 would reduce AF. Beat to beat variation in HR, an index of AF frequency, was analyzed by plotting each RR interval (R) versus the RR interval of the subsequent beat (RI) (n=˜500 beats) (FIG. 7A). An RR interval was the time elapsed between two successive R waves of the QRS signal on an electrocardiogram, and the reciprocal of heart rate. HR beat to beat variation was analyzed at 2 weeks, 4 weeks, and 6 weeks. RR interval showed little beat to beat variation at week 2, varied greatly at week 4, and then returned to a state of less variation at week 6 (FIGS. 7A and 7B). Therefore, AIP×5 was effective in reducing symptoms of AF.

### Example 5: MyoAAV-AIP×5 Suppressed Arrhythmia in Two Different Animal Models of Atrial Fibrillation

Two different atrial fibrillation (AF) mouse models were used to test the efficacy of AIP×5 at treating AF of different origins. The two different AF mouse models were liver kinase B1 (LKB1) knock out mice and T-box transcription factor 5 (Tbx5) knock out mice.

LKB1 encodes a serine/threonine kinase, which functions upstream of the AMP-activated protein kinase (AMPK) superfamily. LKB1 positively regulates the AMP-activated protein kinase (AMPK) and additional AMPK-related downstream kinases. Together, LKB1 and AMPK are involved in cell growth and metabolism and cellular response to energy stress. Tbx5 is part of a group of genes that specify the regions of the body. Tbx5 is involved in the development of the heart, forelimbs, and other structures in the body. It plays a critical role in regulating the formation of the heart and its conduction system. Knockouts of either of these genes in mice induces AF.

LKB1-floxed or Tbx5-floxed mice were used to test the efficacy of AIP×5. AAV-NPPA-Cre was then used to conditionally knock out LKB1 or Tbx5 from the atria of the mice. NPPA is an atria specific promoter, ensuring that Cre recombinase directed knockout of LKB1 or Tbx5 was restricted to the atria. AAV-NPPA-FP (fluorescent protein) was used as a control virus. AAV-NPPA-FP has no Cre, and therefore the gene of interest is not knocked out. Uninjected mice were also included as a control to account for any effects caused by AAV injections. AAV-cTnT-AIP×5 or AAV-cTnT-CN19o were both tested as potential therapies to AF.

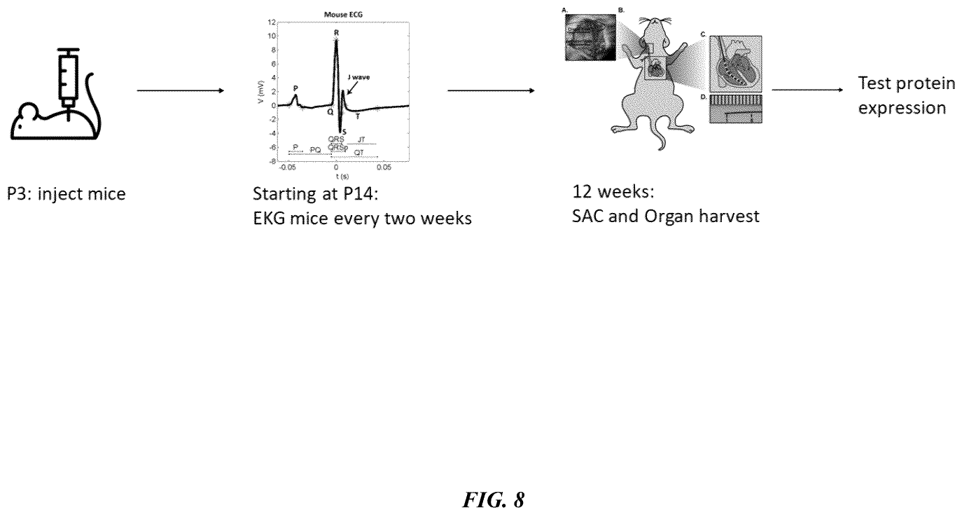

The schematic of the treatment and testing regiment is provided in FIG. 8. In short, P3 LKB1-floxed or Tbx5-floxed mice were injected (or not injected in the uninjected controls), and then EKG measurements of the mice were taken every two weeks starting at P14. At 12 weeks, the mice were sacrificed, and the sacrificed mice were tested for protein expression.

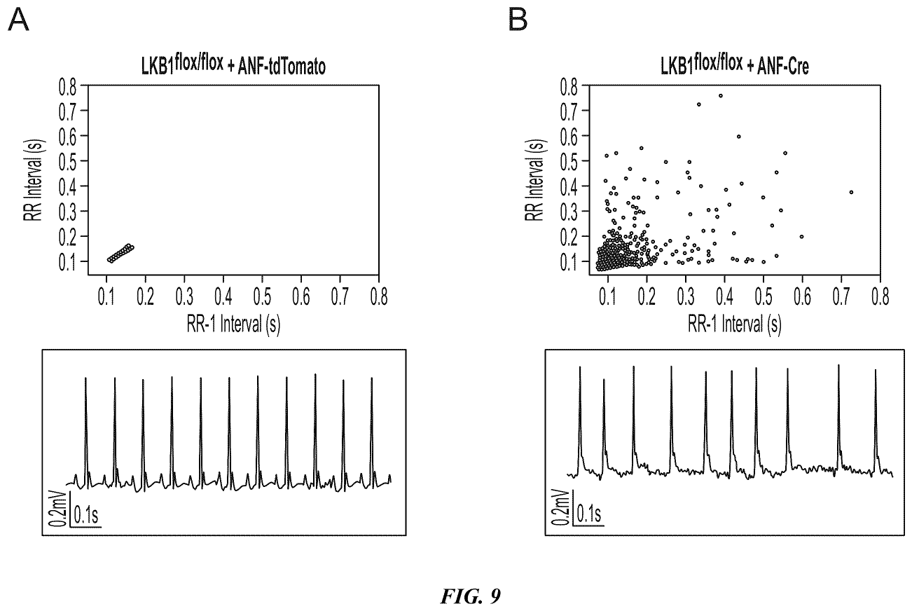

The EKG measurements of the mice were used to calculate whether the mice demonstrated AF. AF was calculated using the standard deviation of the beat-to-beat timing differences. A high beat-to-beat variability when sedated was indicative of atrial misfiring. High beat-to-beat variability was found to be absent in control mice but was present in LKB1 knockout mice as illustrated in FIG. 9.

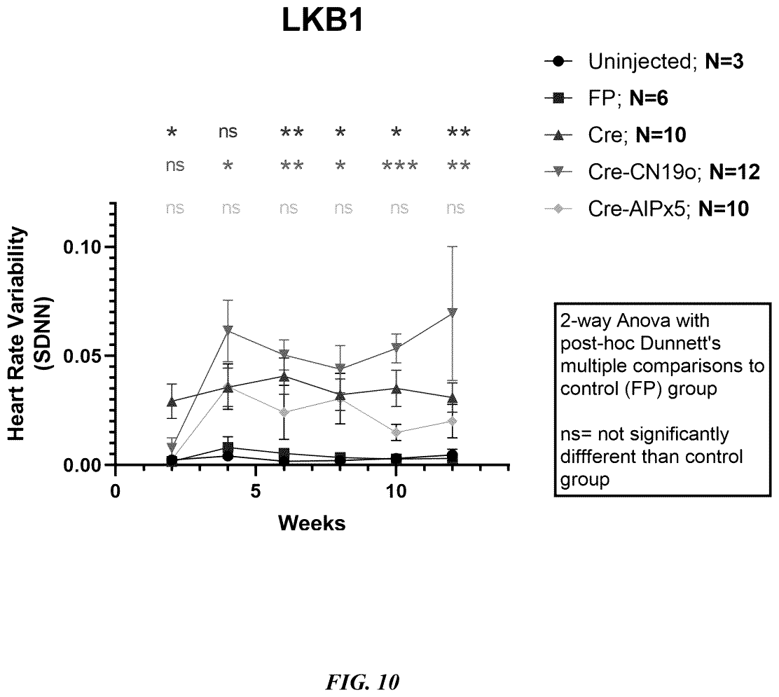

FIG. 10 illustrates that AIP×5 was effective in suppressing beat-to-beat variability in LKB1 knockout mice, and thus suppressing the symptoms of AF. When AIP×5 was present in LKB1 knockout mice, these mice showed no statistically significant difference in beat-to-beat variability as compared to control (non-LKB1 knockout) mice. Surprisingly, CN19o, when provided to LKB1 knockout mice, did not suppress beat-to-beat variability, and in fact performed worse in measures of beat-to-beat variability than LKB1 knockout mice alone.

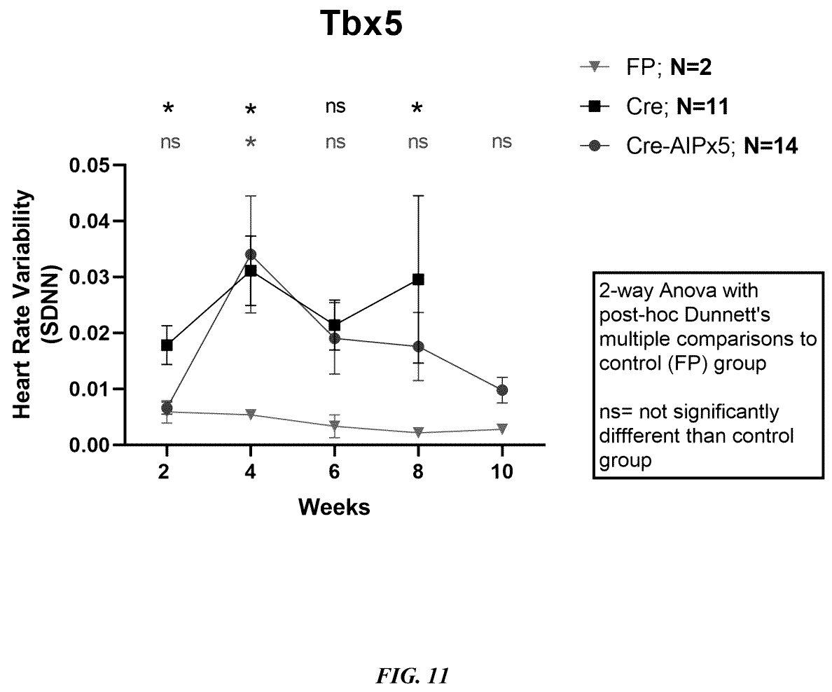

FIG. 11 illustrates that AIP×5 was also effective in suppressing beat-to-beat variability in Tbx5 knockout mice, and thus suppressing the symptoms of AF. When AIP×5 was present in Tbx5 knockout mice, these mice showed no statistically significant difference in beat-to-beat variability after week 4.

Statistical significance in the tests illustrated in FIGS. 10 and 11 was conducted using a 2-way Anova with post-hoc Dunnett's multiple comparisons to control (FP) group.

### OTHER EMBODIMENTS

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A multimeric polypeptide that inhibits CaMKII.

2. The multimeric polypeptide of claim 1, wherein the multimeric polypeptide comprises from about 2 to about 20 repeats of an AIP peptide.

3. The multimeric polypeptide of claim 2, wherein the AIP repeats are contiguous and/or separated by linkers.

4. The multimeric polypeptide of claim 1, wherein the multimeric polypeptide comprises a sequence having at least 85% amino acid sequence identity to:
AIPx3
(SEQ ID NO: 3)
YKKALHRQEAVDALYKKALHRQEAVDALYKKALHRQEAVDAL;
or
AIPx5
(SEQ ID NO: 4)
YKKALHRQEAVDALYKKALHRQEAVDALYKKALHRQEAVDALYKKALH
RQEAVDALYKKALHRQEAVDAL.

5. The multimeric polypeptide of claim 1, wherein the multimeric polypeptide is fused to a 12.6-kDa FK506-binding protein (FKBP12.6) polypeptide.

6. The multimeric polypeptide of claim 1, wherein the EC50 for inhibition of CaMKII by the multimeric polypeptide is less than 10% of the EC50 for AIP.

7. An expression vector comprising a polynucleotide encoding the multimeric polypeptide of claim 1.

8. The expression vector of claim 7, wherein the multimeric polypeptide is operably linked to a promoter suitable for driving expression of the multimeric polypeptide in a mammalian cardiac cell.

9. A pharmaceutical composition comprising an effective amount of the multimeric polypeptide of claim 1.

10. A pharmaceutical composition comprising an effective amount of the expression vector of claim 7.

11. A cell comprising the expression vector of claim 7.

12. A method for modulating a cardiac arrhythmia in a subject, the method comprising contacting a cell in the subject comprising a cardiac ryanodine channel (RYR2) with the multimeric polypeptide of claim 1, or a polynucleotide encoding the multimeric polypeptide.

13. A method for inhibiting phosphorylation of a ryanodine channel (RYR2) polypeptide in a cell, the method comprising contacting a cell comprising a cardiac ryanodine channel (RYR2) with the multimeric polypeptide of claim 1, or a polynucleotide encoding the multimeric polypeptide.

14. A method of treating a subject comprising a mutation associated with a cardiac arrhythmia, the method comprising administering to the subject the multimeric polypeptide of claim 1, or a polynucleotide encoding the multimeric polypeptide.

15. The method of claim 14, wherein the mutation is in a cardiac ryanodine channel (RYR2).

16. The method of claim 15, wherein the mutation is RYR2^{R4651I}.

17. A method of treating a subject having a cardiac disease, condition, or disorder characterized by a cardiac arrhythmia, comprising administering a multimeric AIP polypeptide or a polynucleotide encoding said polypeptide to the subject.

18. A method of treating a subject having a cardiac disease, condition, or disorder characterized by a cardiac arrhythmia, comprising administering an effective amount of an adeno-associated viral vector comprising a polynucleotide encoding a multimeric AIP polypeptide to the subject.

19. The method of claim 18, wherein the effective amount is between about 1×10¹⁰ viral genomes/kg and about 1×10¹⁴ viral genomes/kg.

20. A method of reducing cardiac variability in a subject having atrial fibrillation, comprising administering a multimeric AIP polypeptide or a polynucleotide encoding said multimeric AIP polypeptide to the subject.


SEQUENCE LISTING (15 sequences)

SEQ ID NO: 1 - protein, 14 aa, synthetic construct
YKKALRRQEAVDAL

SEQ ID NO: 2 - protein, 19 aa, synthetic construct
KRAPKLGQIGRQKAVDIED

SEQ ID NO: 3 - protein, 42 aa, synthetic construct
YKKALHRQEAVDALYKKALHRQEAVDALYKKALHRQEAVDAL

SEQ ID NO: 4 - protein, 70 aa, synthetic construct
YKKALHRQEAVDALYKKALHRQEAVDALYKKALHRQEAVDALYKKALHRQEAVDALYKKALHRQEAVDAL

SEQ ID NO: 5 - protein, 13 aa, synthetic construct
KKALRRQEAVDAL

SEQ ID NO: 6 - protein, 13 aa, synthetic construct
KKKLRRQEAFDAL

SEQ ID NO: 7 - protein, 478 aa, Homo sapiens
MASTTTCTRFTDEYQLFEELGKGAFSVVRRCMKIPTGQEYAAKIINTKKLSARDHQKLEREARICRLLKHPNIVRLHDSISEEGFHYLVFDLVTGGELFEDIVAREYYSEADASHCIQQILESVNHCHLNGIVHRDLKPENLLLASKSKGAAVKLADFGLAIEVQGDQQAWFGFAGTPGYLSPEVLRKDPYGKPVDMWACGVILYILLVGYPPFWDEDQHRLYQQIKAGAYDFPSPEWDTVTPEAKDLINKMLTINPAKRITASEALKHPWICQRSTVASMMHRQETVDCLKKFNARRKLKGAILTTMLATRNFSAAKSLLKKPDGVKESTESSNTTIEDEDVKARKQEIIKVTEQLIEAINNGDFEAYTKICDPGLTAFEPEALGNLVEGMDFHRFYFENALSKSNKPIHTIILNPHVHLVGDDAACIAYIRLTQYMDGSGMPKTMQSEETRVWHRRDGKWQNVHFHRSGSPTVPIK

SEQ ID NO: 8 - DNA, 1437 bp, Homo sapiens
  source 1..1437: mol_type = genomic DNA
atggcttcgaccacaacctgcaccaggttcacggacgagtatcagcttttcgaggagcttggaaagggggcattctcagtggtgagaagatgtatgaaaattcctactggacaagaatatgctgccaaaattatcaacaccaaaaagctttctgctagggatcatcagaaactagaaagagaagctagaatctgccgtcttttgaagcaccctaatattgtgcgacttcatgatagcatatcagaagagggctttcactacttggtgtttgatttagttactggaggtgaactgtttgaagacatagtggcaagagaatactacagtgaagctgatgccagtcattgtatacagcagattctagaaagtgttaatcattgtcacctaaatggcatagttcacagggacctgaagcctgagaatttgcttttagctagcaaatccaagggagcagctgtgaaattggcagactttggcttagccatagaagttcaaggggaccagcaggcgtggtttggttttgctggcacacctggatatctttctccagaagttttacgtaaagatccttatggaaagccagtggatatgtgggcatgtggtgtcattctctatattctacttgtggggtatccacccttctgggatgaagaccaacacagactctatcagcagatcaaggctggagcttatgattttccatcaccagaatgggacacggtgactcctgaagccaaagacctcatcaataaaatgcttactatcaaccctgccaaacgcatcacagcctcagaggcactgaagcacccatggatctgtcaacgttctactgttgcttccatgatgcacagacaggagactgtagactgcttgaagaaatttaatgctagaagaaaactaaagggtgccatcttgacaactatgctggctacaaggaatttctcagcagccaagagtttgttgaagaaaccagatggagtaaaggagtcaactgagagttcaaatacaacaattgaggatgaagatgtgaaagcacgaaagcaagagattatcaaagtcactgaacaactgatcgaagctatcaacaatggggactttgaagcctacacaaaaatctgtgacccaggccttactgcttttgaacctgaagctttgggtaatttagtggaagggatggattttcaccgattctactttgaaaatgctttgtccaaaagcaataaaccaatccacactattattctaaaccctcatgtacatctggtaggggatgatgccgcctgcatagcatatattaggctcacacagtacatggatggcagtggaatgccaaagacaatgcagtcagaagagactcgtgtgtggcaccgccgggatggaaagtggcagaatgttcattttcatcgctcggggtcaccaacagtacccatcaagtaa

SEQ ID NO: 9 - protein, 57 aa, synthetic construct
KRAPKLGQIGRQKAVDIEDKRAPKLGQIGRQKAVDIEDKRAPKLGQIGRQKAVDIED

SEQ ID NO: 10 - protein, 95 aa, synthetic construct
KRAPKLGQIGRQKAVDIEDKRAPKLGQIGRQKAVDIEDKRAPKLGQIGRQKAVDIEDKRAPKLGQIGRQKAVDIEDKRAPKLGQIGRQKAVDIED

SEQ ID NO: 11 - protein, 108 aa, Homo sapiens
MGVEIETISPGDGRTFPKKGQTCVVHYTGMLQNGKKFDSSRDRNKPFKFRIGKQEVIKGFEEGAAQMSLGQRAKLTCTPDVAYGATGHPGVIPPNATLIFDVELLNLE

SEQ ID NO: 12 - DNA, 327 bp, Homo sapiens
  source 1..327: mol_type = genomic DNA
atgggcgtggagatcgagaccatctcccccggagacggaaggacattccccaagaagggccaaacgtgtgtggtgcactacacaggaatgctccaaaatgggaagaagtttgattcatccagagacagaaacaaacctttcaagttcagaattggcaaacaggaagtcatcaaaggttttgaagagggtgcagcccagatgagcttggggcagagggcgaagctgacctgcacccctgatgtggcatatggagccacgggccaccccggtgtcatccctcccaatgccaccctcatctttgacgtggagctgctcaacttagagtga

SEQ ID NO: 13 - protein, 149 aa, Homo sapiens
LYELLAALIRGNRKNCAQFSGSLDWLISRLERLEASSGILEVLHCVLVESPEALNIIKEGHIKSIISLLDKHGRNHKVLDVLCSLCVCHGVAVRSNQHLICDNLLPGRDLLLQTRLVNHVSSMRPNIFLGVSEGSAQYKKWYYELMVDH

SEQ ID NO: 14 - DNA, 447 bp, Homo sapiens
  source 1..447: mol_type = genomic DNA
ctgtatgagttgctggcggctctaattagaggaaatcgtaaaaactgtgctcaattttctggctccctcgactggttgatcagcagattggaaagactggaagcttcttcaggcattctggaagttttacactgtgttttagtagaaagtccagaagctctaaatattattaaagaaggacatattaaatctattatctcacttttagacaaacatggaagaaatcacaaggttctggatgtcttgtgctcactctgtgtttgccacggggttgcagtccgttctaaccagcatctcatctgtgacaatctcctaccaggaagagacttgttattgcagacacgtcttgtgaaccatgtcagcagcatgagacccaatatttttctgggcgtcagtgaaggttctgctcagtataagaaatggtactatgaattgatggtggaccac

SEQ ID NO: 15 - protein, 14 aa, synthetic construct
YKKALHRQEAVDAL